(19) United States
(12) Patent Application Publication
(10) Pub. No.: US 2024/0025990 A1
(43) Pub. Date: Jan. 25, 2024

(54) PREVENTION AND TREATMENT OF GRAFT-VERSUS-HOST DISEASE (GVHD)

(71) Applicant: CITY OF HOPE, Duarte, CA (US)

(72) Inventors: Defu ZENG, Duarte, CA (US); Arthur D. RIGGS, Duarte, CA (US); Qingxiao SONG, Duarte, CA (US)

(73) Assignee: CITY OF HOPE, Duarte, CA (US)

(21) Appl. No.: 18/255,503

(22) PCT Filed: Dec. 1, 2021
(86) PCT No.: PCT/US2021/061421

§ 371 Date: Jun. 1, 2023

Related U.S. Application Data
(60) Provisional application No. 63/119,919, filed on Dec. 1, 2020.

(51) Int. Cl. C07K 16/24; A61P 37/06

(52) U.S. Cl. CPC C07K 16/246; A61P 37/06; A61K 2039/505

(57) ABSTRACT

Disclosed is a method of preventing or treating GVHD while preserving GVL activity in a subject receiving a hematopoietic cell transplantation (HCT) by administering to the subject an effective amount of an anti-IL-2 antibody such as an anti-IL-2-JES6 antibody.

### PRIORITY CLAIM

This application claims priority to U.S. Provisional Patent Application No. 63/119,919, filed Dec. 1, 2020, which is incorporated herein by reference in its entirety, including drawings.

### STATEMENT REGARDING FEDERALLY SPONSORED RESEARCH

This invention was made with government support under Grant Numbers R01 A1066008 and R01 CA228465, awarded by the National Institutes of Health. The government has certain rights in the invention.

### BACKGROUND

The success of allogeneic hematopoietic cell transplantation (Allo-HCT) for treatment of hematologic malignancies depends partly on the ability of donor T cells to eliminate residual malignant cells in the recipient after the pre-transplant conditioning, but the same donor T cells also mediate graft-versus-host-disease (GVHD) [1]. Prevention of GVHD in patients with immunosuppressants also suppresses graft-versus-leukemia (GVL) activity [2-4]. Development of approaches that prevent GVHD while preserving GVL activity remains a long-sought goal [5-8].

Interactions of PD-L1 with PD-1 and CD80 on activated T cells have an important role in regulating immune responses [9-11]. Tumor cell PD-L1 interaction with PD-1 on activated T cells tolerizes anti-tumor T cells and prevents anti-tumor immunity [12]. Similarly, recipient tissue PD-L1 interactions with PD-1 and CD80 on alloactivated donor CD8⁺ T cells markedly reduce GVHD severity, although this mechanism is not effective when the graft contains both CD4+ and CD8⁺ T cells [13]. One possible reason is that IL-2 produced by CD4⁺ T cells could prevent tolerance induction by PD-1 signaling [14], although previous studies showed that administration of high-dose IL-2 early after Allo-HCT prevents acute GVHD (aGVHD) while preserving GVL activity [15].

Regulatory T cells including FoxP3⁺ Treg and FoxP3-IL-10⁺ Tr1 cells play important roles in ameliorating aGVHD [16-22]. PD-L1 interaction with PD-1 augments conversion of activated T cells into Foxp3⁺ Treg cells [23]. In the pathogenesis of aGVHD, most regulatory T cells are Tr1 cells that require Eomes for their development [17]. Although PD-L1/PD-1 interaction upregulates expression of Eomes and Blimp-1 during induction of anergy and exhaustion of T effector cells, the role of PD-L1 on Tr1 cell development remains unclear. In addition, persistence of donor CD8⁺ T cell-induced GVHD was mediated by CD8⁺ T memory progenitors (Tmp) [24] that play a critical role in tumor immunity [25].

Accordingly, there is a need to develop an approach that effectively prevents graft-versus-host disease (GVHD) while preserving strong graft-versus-leukemia (GVL) activity in patients. This disclosure satisfies the need in the art.

### SUMMARY

This disclosure is directed to a method of preventing or treating GVHD while preserving GVL activity in vivo in a subject receiving a hematopoietic cell transplantation (HCT). The method entails administering to the subject an effective amount of an anti-IL-2 antibody. In certain embodiments, the subject receives an allogeneic HCT. In certain embodiments, the GVHD is acute GVHD. In certain embodiments, the anti-IL-2 antibody augments IL-2 binding to IL-2Rα and blocks IL-2 binding to IL-2Rβ in vivo. In certain embodiments, the anti-IL-2 antibody is a monoclonal antibody. In certain embodiments, the anti-IL-2 antibody is a recombinant antibody. In certain embodiments, the anti-IL-2 antibody is a human antibody. In certain embodiments, the anti-IL-2 antibody is a humanized antibody. In certain embodiments, the anti-IL-2 antibody is anti-IL-2-JES6 antibody. In certain embodiments, the anti-IL-2 antibody is administered to the subject on the same day of receiving HCT. In certain embodiments, the anti-IL-2 antibody is administered to the subject after receiving HCT. In certain embodiments, multiple doses of the anti-IL-2 antibody are administered after HCT. In certain embodiments, a single dose of the anti-IL-2 antibody is administered each day. In certain embodiments, the anti-IL-2 antibody is administered every other day for a week, for two weeks, for three weeks, or fora month after HCT. In certain embodiments, the subject is human.

In a related aspect, disclosed herein is a composition for use in preventing or treating GVHD while preserving GVL activity in a subject receiving a hematopoietic cell transplantation (HCT). The composition comprises an effective amount of an anti-IL-2 antibody. In certain embodiments, the subject receives an allogeneic HCT. In certain embodiments, the GVHD is acute GVHD. In certain embodiments, the anti-IL-2 antibody augments IL-2 binding to IL-2Rα and blocks IL-2 binding to IL-2Rβ in vivo. In certain embodiments, the anti-IL-2 antibody is a monoclonal antibody. In certain embodiments, the anti-IL-2 antibody is a recombinant antibody. In certain embodiments, the anti-IL-2 antibody is a human antibody. In certain embodiments, the anti-IL-2 antibody is a humanized antibody. In certain embodiments, the anti-IL-2 antibody is anti-IL-2-JES6 antibody. In certain embodiments, the composition comprising the anti-IL-2 antibody is administered to the subject on the same day of receiving HCT. In certain embodiments, the composition comprising the anti-IL-2 antibody is administered to the subject after receiving HCT. In certain embodiments, multiple doses of the composition comprising the anti-IL-2 antibody are administered after HCT. In certain embodiments, a single dose of the composition comprising the anti-IL-2 antibody is administered each day. In certain embodiments, the composition comprising the anti-IL-2 antibody is administered every other day for a week, for two weeks, for three weeks, or fora month after HCT. In certain embodiments, the subject is human.

### BRIEF DESCRIPTION OF THE DRAWINGS

FIG. 1 shows that tolerogenic anti-IL-2 mAb (JES6-1) prevents acute GVHD while preserving strong GVL activity. After administration of tolerogenic anti-IL-2 mAb (JES6-1) that forms anti-IL-2/IL-2 complexes that augment IL-2 binding to IL-2Rα but block IL-2-binding to IL-2Rβ, activated donor T cells increase expression of PD-1 and Tcf1 (Ly108) and decrease production of GM-CSF. Some donor CD8⁺ T cells differentiate into PD-1^{hi}Ly108⁺ T effector memory (Tem) progenitors. In the lymphoid tissues, the Tem progenitors and their derivatives Teff are well preserved due to lack of PD-1 interaction with PD-L1 expressed by host-tissues, leading to strong GVL activity. In contrast, in the GVHD target tissues, plenty of host-tissue PD-L1 interaction with PD-1 on donor T cells results in depletion of GM-CSF⁺ Th1/Tc1 as well as expansion of Foxp3⁺ Treg and Foxp3⁻ Tr1 cells, leading to effective prevention of acute GVHD.

FIGS. 2A-2G show that tolerogenic anti-IL-2 mAb (JES6-1A12) but not non-tolerogenic anti-IL2 mAb (S4B6) prevents acute GVHD and preserves GVL activity more effectively than tacrolimus. FIGS. 2A-2D: Lethally irradiated WT BALB/c recipients were given splenocytes (5×10⁶) with or without TCD-BM (2.5×10⁶) from WT C57BL/6 donors. Recipients were given a total of 4 i.p. injections of rat-IgG, anti-IL-2 mAb (JES6-1A12) or anti-IL-2 mAb (S4B6) (500 μg/mouse) at days 0, 2, 4 and 6 after HCT. FIGS. 2A and 2C: Plots of % Original Body weight, diarrhea and % survival are shown. n=7-8 combined from 2 replicated experiments. FIGS. 2B and 2D: Mean±SEM of histopathology scores of liver, small intestine and colon are shown; n=4 per group. Combined from 2 replicated experiments. FIG. 2E: WT BALB/c recipients given splenocytes and TCD-BM cells from WT C57BL/6 donors and injected IgG or anti-IL-2 mAb (JES6-1A12) as described in FIGS. 2A-2D. Recipients were challenged with i.p. injection of BCL1/Luc cells (5×10⁶/mouse) on day 0. Mice were monitored for tumor growth using in vivo bioluminescent imaging (BLI), clinical signs of GVHD and survival. One representative BLI image from each time point is shown for each group and summary of photons/sec, diarrhea and tumor-free survival of recipients. n=8 combined from 2 replicated experiments. FIG. 2F: Lethally irradiated WT BALB/c recipients were given splenocytes (2.5×10⁶) and TCD-BM (2.5×10⁶) from WT C57BL/6 donors. Recipients were challenged with i.p. injection of BCL1/Luc cells (5×10⁶/mouse) on day 0 and were given a total of 4 i.p. injections anti-IL-2 mAb (JES6-1A12) (500 μg/mouse) at days 0, 2, 4 and 6 after HCT, or daily i.p. injections of tacrolimus (0.75 mg/Kg) at days 0-21 after HCT. One representative BLI image from each time point is shown for anti-IL-2 mAb (JES6-1A12) and tacrolimus group and summary of photons/sec, body weight change, as well as the tumor free survival of recipients. n=8-10 combined from 2 replicated experiments. FIG. 2G: Lethally irradiated BALB/c recipients were given splenocytes (1.25×10⁶) and TCD-BM (2.5×10⁶) from C57BL/6 donors. Recipients were challenged with i.p. injection of BCL1/Luc cells (10×10⁶/mouse) on day 0 and were given a total of 4 i.v. injections IL-2 mAb (500 μg/mouse) at days 0, 2, 4 and 6 after HCT, or i.p. injections of tacrolimus (0.75 mg/Kg) once daily until moribund with tumor growth. One representative BLI image from each time point is shown for IL-2 mAb (JES6-1A12) and tacrolimus group and summary of photons/sec and tumor-free survival of recipients. n=8-10 combined from 2 replicated experiments. “+” indicates death. Data represent mean±SE. P values were calculated by ordinary one-way ANOVA (FIGS. 2B & 2D), two-tailed Student t tests (FIGS. 2F & 2G) or log-rank test for survival comparison (FIGS. 2A, 2E, 2F and 2G) (*p<0.05, ** p<0.01, *** p<0.001, **** p<0.0001).

FIGS. 3A and 3B show prevention of aGVHD by tolerogenic anti-IL-2 mAb (JES6-1A12) but not by non-tolerogenic anti-IL-2 mAb (S4B6). Lethally irradiated WT BALB/c recipients were given splenocytes (5×10⁶) and TCD-BM (2.5×10⁶) from C57BL/6 donors. Recipients were given a total of 4 i.v. injections of control IgG, anti-IL-2 mAb (JES6-1A12) or anti-IL-2 mAb (S4B6) (500 μg/mouse) at days 0, 2, 4 and 6 after HCT. At 7 days after HCT, histopathology of liver, small intestine and colon was evaluated. A representative photomicrograph is shown, original magnification ×100 (liver), ×200 (small intestine and colon).

FIGS. 4A and 4B show that tolerogenic anti-IL-2 mAb (JES6-1A12) preserves GVL activity against BC-CML tumor cell. Lethally irradiated C57BL/6 recipients were given TCD-BM (2.5×10⁶) with or without splenocytes (2.5×10⁶) from A/J donors. Recipients were challenged with i.v. injection of GFP+BC-CML cells (1×10⁶/mouse) at day 0 and were given at total of 4 i.v. injections anti-IL-2 mAb (JES6-1A12) (500 μg/mouse) or control IgG at days 0, 2, 4 and 6 after HCT. FIG. 4A: Plots of % Original body weight, % mice without diarrhea and % survival. n=8 from two replicate experiments. FIG. 4B: Moribund mice with or without GVHD during observation and mice at day 40 after HCT were checked for BC-CML tumor cells in the spleen, liver and bone marrow. Representative flow cytometry patterns and percentages of BC-CML cells in the spleen, liver and bone marrow from each group are shown. n=6 combined from duplicated experiments. Data represent mean±SE. Nonlinear regression (curve fit) was used for body weight comparisons. Log-rank test was used for survival comparisons. 2-way ANOVA was used for BC-CML cell comparison (*p<0.05, ** p<0.01, **** p<0.0001).

FIGS. 5A-5D show that tolerogenic anti-IL-2 mAb (JES6-1A12) augments NK cells expansion. FIGS. 5A-5B: Lethally irradiated WT BALB/c recipients were given splenocytes (5×10⁶) and TCD-BM (2.5×10⁶) from WT C57BL/6 donors. Recipients were given a total of 4 i.v. injections of control IgG or anti-IL-2 mAb (JES6-1A12) (500 μg/mouse) at days 0, 2, 4 and 6 after HCT. At day 6 after HCT, NK cells were enumerated in the spleen and liver. Representative flow cytometry pattern (FIG. 5A), percentage and yield of donor NK cells are shown (FIG. 5B), n=4-6 from 2 replicates. FIGS. 5C-5D: WT BALB/c recipients were engrafted with splenocytes and TCD-BM cells from WT C57BL/6 donors as described in FIG. 2D. Recipients were given a total of 4 i.v. injections of anti-IL-2 mAb (JES6-1A12) (500 μg/mouse) at days 0, 2, 4 and 6 with anti-NK1.1 (200 ug/mouse) or control IgG once a week after HCT. FIG. 5C: Day 24 after HCT, NK cells were enumerated in the spleen and liver from recipients given anti-NK1.1 treatment. Representative flow cytometry patterns are shown. FIG. 5D: Representative BLI image from each time point of one experiment is shown for each group. Summaries of photons/second and % survival of recipients are shown. n=7 combined from duplicated experiments. Data represent mean±SE. P values were calculated by unpaired two-tailed Student t test (* p<0.05, ** p<0.01).

FIGS. 6A-6B show that tolerogenic anti-IL-2 mAb (JES6-1A12) prevents aGVHD and preserves GVL activity more effectively than tacrolimus. Lethally irradiated BALB/c recipients were given splenocytes (1.25×10⁶) and TCD-BM (2.5×10⁶) from C57BL/6 donors. Recipients were challenged with i.p. injection of BCL1/Luc cells (5×10⁶/mouse) at day 0 and were given a total of 4 i.v. injections anti-IL-2 mAb (JES6-1A12) (500 μg/mouse) at days 0, 2, 4 and 6 after HCT or i.p. injections of tacrolimus (0.75 mg/Kg) daily until moribund with tumor growth. FIG. 6A: Representative BLI image from each time point of one experiment is shown for IL-2 mAb and tacrolimus group. FIG. 6B: Summary of photons/second and survival curve of recipients are shown. n=10 combined from duplicated experiments. Data represent mean±SE. P values were calculated by log-rank test (*p<0.05).

FIGS. 7A-7J show that prevention of aGVHD by tolerogenic anti-IL-2 mAb (JES6-1A12) requires PD-L1 expression by GVHD target tissues. Lethally irradiated WT or PD-L1^{-/-} BALB/c recipients were given splenocytes (2.5×10⁶) and TCD-BM (2.5×10⁶) from C57BL/6 donors. Recipients were given a total of 4 i.p. injections of rat-IgG, anti-IL-2 mAb (JES6-1A12) or anti-IL-2 mAb (S4B6) (500 μg/mouse) at days 0, 2, 4 and 6 after HCT. Recipients given TCD-BM cells (2.5×10⁶) alone were used as controls. FIG. 7A: Plots of % original body weight, diarrhea and % survival are shown. n=8 per group. Combined from 2 replicated experiments. FIG. 7B: Mean±SEM of histopathology scores of liver, small intestine and colon are shown; n=4 per group. Combined from 2 replicated experiments. FIGS. 7C-7F: At day 6 after HCT, spleen, liver and colon of recipients were harvested for analysis of donor CD4⁺ and CD8⁺ T cell percentage and yield. Mean±SEM of the percentage and yield of H-2K^{b+} TCRβ⁺ CD4⁺ or CD8⁺ T cells are shown; n=4-11 per group. Combined from two to three replicated experiments. FIGS. 7G-7I: Day 6 post HCT, percentage of Eomes⁺PD1⁺ cells among donor CD4⁺ and CD8⁺ T cells in liver and colon of WT or PD-L1^{-/-} recipients treated with anti-IL-2 mAb (JES6-1A12), anti-IL-2 mAb (S4B6) or control IgG; n=4-5 per group. FIG. 7J: Day 6 post HCT, percentage of Eomes⁺PD1⁺ cells among donor CD4⁺ and CD8⁺ T cells in liver and colon of WT and PD-L1^{-/-} recipients treated with anti-IL-2 mAb (JES6-1A12); n=4 per group. Data represent mean±SE combined from two replicated experiments. P values were calculated by log-rank test (FIG. 7A) or one-way ANOVA (FIG. 7B) or 2-way ANOVA (FIGS. 7C-7J) (*p<0.05, **p<0.01, ***p<0.001, **** p<0.0001).

FIG. 8 shows that prevention of aGVHD by tolerogenic anti-IL-2 mAb (JES6-1A12) requires PD-L1 expression by GVHD target tissues. Lethally irradiated WT or PD-L1^{-/-} BALB/c recipients were given splenocytes (5×10⁶) and TCD-BM (2.5×10⁶) from C57BL/6 donors. Recipients were given a total of 4 i.v. injections of control IgG or anti-IL-2 mAb (JES6-1A12) (500 μg/mouse) at days 0, 2, 4 and 6 after HCT. At 7 days after HCT, histopathology of liver, small intestine and colon was evaluated. A representative photomicrograph is shown, original magnification ×100 (liver), ×200 (small intestine and colon).

FIGS. 9A-9D show that prevention of GVHD by tolerogenic anti-IL-2 mAb (JES6-1A12) but not non-tolerogenic anti-IL-2 mAb (S4B6) is associated with tissue PD-L1-dependent depletion of GM-CSF-producing Th1 and Tc1 cells. Lethally irradiated WT and PD-L1^{-/-} BALB/c recipients were given splenocytes (2.5×10⁶) and TCD-BM (2.5×10⁶) from C57BL/6 donors. Recipients were given a total of 3 i.p. injections of rat-IgG, anti-IL-2 mAb (JES6-1A12) or anti-IL-2 mAb (S4B6) (500 μg/mouse) at days 0, 2, and 4 after HCT. On day 6, donor cells in the spleen, liver and colon were analyzed for cytokine profile. FIGS. 9A-9B: Percentage and yield of GM-CSF⁺ cells among donor IFN-γ+CD4⁺ and CD8⁺ T cells in spleen, liver and colon of WT recipients treated with anti-IL-2 mAb (JES6-1A12 or S4B6) or control IgG. FIG. 9C: Percentage and yield of GM-CSF⁺ cells among donor IFN-γ⁺ CD4⁺ and CD8⁺ T cells in spleen, liver and colon of PD-L1^{-/-} recipients treated with anti-IL-2 mAb (JES6-1A12) or control IgG; n=4 per group. FIG. 9D: Percentage and yield of GM-CSF⁺ cells among donor IFN-γ⁺ CD4⁺ and CD8⁺ T cells in spleen, liver and colon of WT or PD-L1^{-/-} recipients treated with anti-IL-2 mAb (JES6-1A12); n=5 per group. Data represent mean±SE combined from two replicate experiments. P values were calculated by unpaired two-tailed Student t tests (*p<0.05, ** p<0.01, ***p<0.001 **** p<0.0001).

FIG. 10 shows representative flow cytometry patterns of FIG. 9A.

FIG. 11 shows representative flow cytometry patterns of FIG. 9B.

FIG. 12 shows representative flow cytometry patterns of FIG. 9C.

FIG. 13 shows representative flow cytometry patterns of FIG. 9D.

FIGS. 14A-14J show that tolerogenic anti-IL-2 mAb (JES6-1A12) results in the reduction of donor myeloid cells. Lethally irradiated WT BALB/c recipients were given splenocytes (2.5×10⁶) and TCD-BM (2.5×10⁶) from C57BL/6 donors. Recipients were given a total of 4 i.v. injections of control IgG or anti-IL-2 mAb (JES6-1A12) (500 μg/mouse) at days 0, 2, 4 and 6. At day 12 after HCT, cells from the spleen (SPL), liver and colon were analyzed by flow cytometry. FIGS. 14A-14B: Example gating (14A) and quantification of myeloid cell populations (14B) in the liver. FIGS. 14C-14D: Example gating (14C) and frequency of pro-IL-1p-producing neutrophils and monocytes within the H2Kb⁺ CD45⁺ population from liver (14D). FIGS. 14E-14F: Example gating (14E) and frequency of pro-IL-1p-producing neutrophils and monocytes within the H2Kb⁺ CD45⁺ population from colon (14F). FIGS. 14G-14H: Example gating (14G) and frequency of pro-IL-1p-producing neutrophils and monocytes within the H2Kb⁺ CD45⁺ population from spleen (14H). FIGS. 141-14J: Flow cytometric analysis of ROS (CellROX reagent) in the spleen showing representative histograms (14I) and quantification of median fluorescence intensity (MFI) for neutrophils, monocytes, and MDCs (14J). Data represent two experiment with n=4 to 6 per group. Data represent mean±SE. P values were calculated by unpaired two-tailed Student t tests (*p<0.05, **p<0.01).

FIGS. 15A-15C show that tolerogenic (JES6-1A12) and non-tolerogenic (S4B6) anti-IL-2 mAb exhibit different impact on Foxp3⁺ Treg cells. Lethally irradiated WT (FIGS. 15A-15B) or PD-L1^{-/-} BALB/c (FIG. 15C) recipients were given splenocytes (2.5×10⁶) and TCD-BM (2.5×10⁶) from C57BL/6 donors. Recipients were given a total of 3 i.v. injections of rat-IgG or 500 μg/mouse anti-IL-2 mAb (JES6-1A12) (15A) or anti-IL-2 mAb (S4B6) (15B) at days 0, 2, and 4 after HCT. At day 6 after HCT, FoxP3⁺ Treg cells were measured in the spleen, liver and colon. n=4-5 per group. Combined from two experiments. Data represent mean±SE. P values were calculated by 2-way ANOVA (* p<0.05, **p<0.01).

FIGS. 16A-16G show that prevention of acute GVHD by tolerogenic anti-IL-2 mAb (JES6-1A12) but not non-tolerogenic anti-IL-2 mAb (S4B6) requires PD-L1-dependent expansion of Tr1 cells. FIGS. 16A-16E: Lethally irradiated WT or PD-L1^{-/-} BALB/c recipients were given splenocytes (2.5×10⁶) and TCD-BM (2.5×10⁶) from C57BL/6 donors. Recipients were given a total of 3 i.p. injections of rat-IgG or anti-IL-2 mAb (JES6-1A12 or S4B6) (500 μg/mouse) at days 0, 2, and 4 after HCT. Day 6 after HCT, donor-type T cells from live and colon were analyzed for Tr1 cells. FIGS. 16A-16B: Representative staining pattern with percentage and yield of donor IL-10⁺ Foxp3⁻ CD4⁺ T cells in liver and colon of WT recipients treated with anti-IL-2 mAb (JES6-1A12 or S4B6) or control IgG; n=5 per group. FIG. 16C: Representative staining pattern with percentage and yield of donor IL-10⁺ Foxp3⁻ CD4⁺ T cells in liver and colon of PD-L1^{-/-} recipients treated with anti-IL-2 mAb (JES6-1A12) or control IgG; n=4 per group. FIGS. 16D-16E: Blimp-1 and Eomes expression on of donor CD4⁺ T cells in liver and colon of WT recipients treated with anti-IL-2 mAb (JES6-1A12 or S4B6) or control IgG; n=4 per group. FIG. 16F: Blimp-1 and Eomes expression on of donor CD4⁺ T cells in liver and colon of PD-L1^{-/-} recipients treated with anti-IL-2 mAb (JES6-1A12) or control IgG; n=4 per group. FIG. 16G: Lethally irradiated WT BALB/c recipients were given T cells (1.0×10⁶) from WT or IL-10^{-/-} C57BL/6 donors and TCD-BM (2.5×10⁶) from WT C57BL/6 donors. Recipients were given a total of 4 i.p. injections of anti-IL-2 mAb (JES6-1A12) (500 μg/mouse) at days 0, 2, 4 and 6 after HCT, plots of % original body weight, diarrhea and survival are shown. n=8 per group. Data represent mean±SE combined from two replicate experiments. P values were calculated by 2way ANOVA (FIGS. 16A-16F) or log-rank test (FIG. 16G) (*p<0.05, ** p<0.01, ***p<0.001, **** p<0.0001).

FIG. 17 shows that H2Kb⁺TCRβ⁺ IL-10⁺FoxP3⁻ CD4⁺ Tr1 cells are IFN-γ⁺ but GM-CSF⁻. One representation is shown from four replicate experiments.

FIG. 18 shows that IL-10 deficiency in donor T cells does not affect the percentages or yields of GM-CSF⁺ Th/Tc1 cells. Lethally irradiated WT BALB/c recipients were given T cells (1.0×10⁶) from WT or IL-10^{-/-} C57BL/6 donors with TCD-BM (2.5×10⁶) from WT C57BL/6 donors. On day 6 after HCT, GM-CSF+Th/Tc1 cells were enumerated in the spleen, liver and colon by flow cytometry. % and yield of GM-CSF+Th/Tc1 are shown. n=4 per group. Data represent mean±SE combined from two replicate experiments.

FIG. 19 shows that tolerogenic anti-IL-2 mAb (JES6-1A12) decreases the percentage of donor G-MDSC in the liver. Lethally irradiated WT BALB/c recipients were given splenocytes (2.5×10⁶) and TCD-BM (2.5×10⁶) from C57BL/6 donors. Recipients were given a total of 4 i.v. injections of control IgG or anti-IL-2 mAb (JES6-1A12) (500 μg/mouse) at days 0, 2, 4 and 6. At day 12 after HCT, cells from the spleen (SPL), liver and colon were analyzed by flow cytometry. Percentages of G-MDSC and M-MDSC are shown. n=4, data represent mean±SE combined from two replicate experiments. P values were calculated by 2-way ANOVA (**p<0.01).

FIGS. 20A-20E show that prevention of GVHD by tolerogenic anti-IL-2 mAb (JES6-1A12) but not non-tolerogenic anti-IL-2 mAb (S4B6) requires expression of PD-L1 by GVHD target tissues to inhibit activation of IL-2-Stat-5 and AKT-mTOR pathways in donor T cells. Lethally irradiated WT and PD-L1^{-/-} BALB/c recipients were given splenocytes (2.5×10⁶) and TCD-BM (2.5×10⁶) from C57BL/6 donors. Recipients were given a total of 3 i.v. injections of rat-IgG or anti-IL-2 mAb (JES6-1A12 or S4B6) (500 μg/mouse) at days 0, 2, and 4 after HCT. At day 6 after HCT, spleen and colon were harvested for analysis. FIG. 20A: Representative GSEA plots are shown of IL-2-STAT5 pathway-related gene sets expression on CD4⁺ T and CD8⁺ T cells in the spleen or colon of the WT recipients treated with anti-IL-2 mAb (JES6-1A12) versus IgG cohorts. P values were calculated by bioconductor package “clusterProfiler” v3.10.1, * p<0.05, **p<0.01. FIGS. 20B-20C: pAKT and pMTOR expression on of donor CD4⁺ T cells in spleen and colon of WT recipients treated with anti-IL-2 mAb (JES6-1A12 or S4B6) or control IgG; n=5 per group. FIG. 20D: pAKT and pMTOR expression on of donor CD4⁺ T cells in spleen and colon of PD-L1^{-/-} recipients treated with anti-IL-2 mAb (JES6-1A12) or control IgG; n=4 per group. FIG. 20E: pAKT and pMTOR expression on donor CD4⁺ T cells in spleen and colon of WT or PD-L1^{-/-} recipients treated with anti-IL-2 mAb (JES6-1A12); n=4-5 per group. Data represent mean±SE combined from two replicate experiments. P values were calculated by unpaired two-tailed Student t tests (*p<0.05, **p<0.01, ****p<0.0001).

FIG. 21 shows the comparison of the percentage of host parenchymal cells versus donor T cells. Lethally irradiated WT BALB/c recipients were given splenocytes (2.5×10⁶) and TCD-BM (2.5×10⁶) from C57BL/6 donors. Recipients were given a total of 4 i.v. injections of anti-IL-2 mAb (JES6-1A12) (500 μg/mouse) at days 0, 2, 4 and 6 after HCT. Day 7 after HCT, spleen and colon were harvested for analysis. The percentage of H2Kd⁺ CD45⁻ cells versus H2Kb⁺ TCRβ⁺ cells is shown. n=5 per group. Data represent mean±SE combined from two replicate experiments. P values were calculated by unpaired two-tailed Student t tests (** p<0.01).

FIGS. 22A-22D show that tolerogenic anti-IL-2 (JES6-1A12) but not non-tolerogenic anti-IL-2 (S4B6) treatment inhibits activation of AKT-mTOR pathway in CD8⁺ T cells in a host-tissue PD-L1-dependent manner. Lethally irradiated WT or PD-L1^{-/-} BALB/c recipients were given splenocytes (2.5×10⁶) and TCD-BM (2.5×10⁶) from C57BL/6 donors. Recipients were given a total of 3 i.v. injections of rat-IgG or anti-IL-2 mAb (500 μg/mouse) at days 0, 2, and 4 after HCT. At day 6 after HCT, spleen and colon were harvested for analysis. FIG. 22A: pAKT and pMTOR expression on of donor CD8⁺ T cells in spleen and colon of WT recipients treated with anti-IL-2 (JES6-1A12) or control IgG; n=5 per group. FIG. 22B: pAKT and pMTOR expression on of donor CD8⁺ T cells in spleen and colon of WT recipients treated with anti-IL-2 (S4B6) or control IgG; n=5 per group. FIG. 22C: pAKT and pMTOR expression on of donor CD8⁺ T cells in spleen and colon of PD-L1^{-/-} recipients treated with anti-IL-2 or control IgG; n=4 per group. FIG. 22D: pAKT and pMTOR expression on of donor CD8⁺ T cells in spleen and colon of WT or PD-L1^{-/-} recipients treated with anti-IL-2; n=4-5 per group. Data represent mean±SE combined from two replicate experiments. P values were calculated by 2-way ANOVA (*p<0.05, ** p<0.01, ***p<0.001).

FIGS. 23A-23D show that tolerogenic anti-IL-2 mAb (JES6-1A12) and tacrolimus induce distinct T cell subpopulations and T cell transcriptional signatures. FIG. 23A: t-SNE plot displaying clusters identified in spleen cells from recipients treated with anti-IL-2 mAb (JES6-1A12) or tacrolimus on day 7 after HCT. FIG. 23B: Violin plots showing CD3e, CD4 and CD8a distribution on individual clusters. FIG. 23C: Violin plot showing Tcf7 expression level in individual clusters under anti-IL-2 mAb (JES6-1A12) or tacrolimus treatment. FIG. 23D: Compared T cell percentage in individual clusters under anti-IL-2 mAb (JES6-1A12) or tacrolimus treatment.

FIG. 24 shows that scRNA-Seq reveals distinct subpopulations and transcriptional signatures of CD8 T cells after treatment with tolerogenic anti-IL-2 (JES6-1A12) mAb or tacrolimus. Heatmap showing differentially expressed coding genes for the 9 clusters in FIG. 23A.

FIGS. 25A-25L show that tolerogenic anti-IL-2 mAb (JES6-1A12) preserves CD8⁺ T memory progenitors and functional effectors that mediate GVL activity in lymphoid tissues more effectively than tacrolimus. Lethally irradiated WT BALB/c recipients were given splenocytes (2.5×10⁶) and TCD-BM (2.5×10⁶) from C57BL/6 donors. Recipients were given a total of 4 i.p. injections of anti-IL-2 mAb (JES6-1A12) (500 μg/mouse) at days 0, 2, 4 and 6 after HCT, or once daily i.p. injections of tacrolimus (0.75 mg/Kg) at days 0-6 after HCT. On day 7 after HCT, mesenteric lymph node (MLN), spleen (SPL), liver and colon were harvested for analysis. FIG. 25A: Representative pattern of gating strategy in recipients given anti-IL-2 treatment. FIG. 25B: Representative flow cytometry pattern showing the expression of Granzyme B and CD107a on CD8⁺ T cells and % of CD107a⁺GranzymeB⁺ CD8⁺ T cells in the spleen from IL-2 mAb or tacrolimus-treated recipients are showed, n=4-6, combined from 2 experiments. FIGS. 25C-25H: Percentage of Tmp (CD39-Ly108⁺), Teff (CD39⁺Ly108⁺) and Tex (CD39⁺Ly108⁻) among donor CD8⁺ T cells in MLN, spleen, Liver and colon of WT recipients treated with anti-IL-2 mAb (JES6-1A12), tacrolimus or control IgG; n=4-6 per group. FIG. 25I: Percentage of GranzymeB⁺ CD107a⁺ CD8⁺ T, IFN-γ⁺ CD8⁺ T cells and MFI of perforin among Teff, Tex and Tmp in spleen and MLN of WT recipients treated with anti-IL-2 mAb (JES6-1A12). n=4-6 per group. FIGS. 25J-25K: Expression of T-bet, CD127, BrdU and Tim-3 on Teff, Tex and Tmp in spleen and MLN of WT recipients treated with anti-IL-2 mAb (JES6-1A12). n=4-6 per group. FIG. 25L: Percentage of IL-2⁺ CD4⁺ T cells in spleen and MLN of WT recipients treated with anti-IL-2 mAb (JES6-1A12) or tacrolimus. n=4 per group. P values were calculated by unpaired two-tailed Student t tests, one-way ANOVA or two-way ANOVA (*p<0.05, ** p<0.01, ***p<0.001 **** p<0.0001).

FIG. 26 shows gating strategy showing the Teff, Tex, Tex and Ter-Teff CD8⁺ T cell subsets in the spleen of the WT recipients treated with tacrolimus.

FIGS. 27A-27C show that tolerogenic anti-IL-2 (JES6-1A12) treatment is more effective than TAC treatment for preserving CD8⁺ T memory progenitors and functional effectors that mediate GVL effect in lymphoid tissues. Lethally irradiated WT BALB/c recipients were given splenocytes (2.5×10⁶) and TCD-BM (2.5×10⁶) from C57BL/6 donors. Recipients were given a total of 4 i.v. injections of anti-IL-2 mAb (JES6-1A12) (500 μg/mouse) at days 0, 2, 4 and 6 after HCT or i.v. injections of tacrolimus (0.75 mg/Kg) at days 0-6 after HCT. On day 7 after HCT, mesenteric lymph node (MLN), spleen (SPL), liver and colon were harvested for analysis. FIG. 27A: Yield of total donor CD8⁺ T cells and % of KLRG1⁺ CD39⁺ CD8⁺ T cells in the spleen from anti-IL-2 or tacrolimus-treated recipients are showed, n=4-9, combined from 2 experiments. FIG. 27B: Representative flow cytometry patterns showing the expression of Ly108 and CD39 expression in mesenteric lymph node, spleen, liver and colon from anti-IL-2 or tacrolimus-treated recipients. FIG. 27C: Representative flow cytometry pattern showing CD107a, Granzyme B expression among Teff, Tex, Tmp and Ter-Teff cells in the spleen from anti-IL-2 or tacrolimus-treated recipients.

### DETAILED DESCRIPTION

This disclosure is directed to a method of preventing or treating GVHD while preserving GVL activity in vivo in a subject receiving a hematopoietic cell transplantation (HCT). The method entails administering to the subject an effective amount of an anti-IL-2 antibody. In certain embodiments, the subject receives an allogeneic HCT. In certain embodiments, the GVHD is acute GVHD. In certain embodiments, the anti-IL-2 antibody augments IL-2 binding to IL-2Rα and blocks IL-2 binding to IL-2Rβ in vivo. In certain embodiments, the anti-IL-2 antibody is a monoclonal antibody. In certain embodiments, the anti-IL-2 antibody is a recombinant antibody. In certain embodiments, the anti-IL-2 antibody is a human antibody. In certain embodiments, the anti-IL-2 antibody is a humanized antibody. In certain embodiments, the anti-IL-2 antibody is anti-IL-2-JES6 antibody.

“Treating” or “treatment” of a disease or a condition may refer to preventing the disease or condition, slowing the onset or rate of development of the disease or condition, reducing the risk of developing the disease or condition, preventing or delaying the development of symptoms associated with the disease or condition, reducing or ending symptoms associated with the disease or condition, generating a complete or partial regression of the disease or condition, or some combinations thereof.

As used herein, the term “subject” refers to mammalian subject, preferably a human. The phrases “subject” and “patient” are used interchangeably herein.

An “effective amount,” “therapeutically effective amount” or “effective dose” is an amount of a composition (e.g., an antibody or a pharmaceutical composition) that produces a desired therapeutic effect in a subject, such as preventing or treating a target disease or condition, or alleviating symptoms associated with the disease or condition. The precise therapeutically effective amount is an amount of the composition that will yield the most effective results in terms of efficacy of treatment in a given subject. This amount will vary depending upon a variety of factors, including but not limited to the characteristics of the active agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of an active agent and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, P A, 2005.

The administration schedule and doses of the anti-IL-2 antibody can be determined based on the need of the subject. For example, the anti-IL-2 antibody is administered to the subject immediately before or on the same day of receiving HCT. In certain embodiments, the anti-IL-2 antibody is administered to the subject after receiving HCT. In certain embodiments, the anti-IL-2 antibody is administered to the subject receiving HCT at the onset of GVHD. In certain embodiments, multiple doses of the anti-IL-2 antibody are administered after HCT. In certain embodiments, a single dose of the anti-IL-2 antibody is administered each day. In certain embodiments, the anti-IL-2 antibody is administered every other day for a week, for two weeks, for three weeks, or for a month after HCT. One of ordinary skill in the art would understand that when multiple doses of the anti-IL-2 antibody is administered, each dosage may be the same or different. For example, a higher dosage may be administered immediately after HCT and followed by a lower dosage at a later time, e.g., after a week of administration on every other day. Alternatively, a lower dosage may be administered first, followed by a higher dosage.

Any suitable administration route of the anti-IL-2 antibody may be chosen. For example, the anti-IL-2 antibody can be administered to the subject by intravenous, intradermal, subcutaneous, intramuscular, intraperitoneal, intranodal, or intrasplenic administration.

Administration of anti-IL-2 mAb may not simply neutralizing IL-2 in vivo. Certain anti-IL-2 mAb form complexes with IL-2 (IL-2C) that modulate IL-2 interactions with IL-2Rα and IL-2Rβ [26]. Anti-IL-2 from hybridoma clone JES6-1 (anti-IL-2-JES6) forms an IL-2C that enhances IL-2 interaction with IL-2Rα and augments IL-2Rα^{hi} Foxp3⁺ Treg expansion, while blocking IL-2 interaction with IL-2Rβ on conventional T cells [26, 27]. In contrast, anti-IL-2 from hybridoma clone S4B6 (anti-IL-2-S4B6) forms an IL-2C that blocks IL-2 interaction with IL-2Rα and augments IL-2 interaction with IL-2Rβ, leading to expansion of conventional T cells [26, 27]. Thus, anti-IL-2-JES6 has tolerogenic effects, while anti-IL-2-S4B6 does not [26]. Surprisingly, administration of anti-IL-2-JES6 can prevent GVHD while preserving GVL activity; whereas other anti-IL-2 antibodies may not have such effects. Accordingly, disclosed herein is a novel approach for preventing or treating GVHD in a subject receiving HCT using an antibody that preferentially blocks IL-2-binding to IL-2Rβ and augments IL-2-binding to IL-2Rα such that GVHD is prevented or treated while strong GVL effect is preserved. Administration of the anti-IL-2 antibody depletes pathogenic GM-CSF-producing Th1/Tc1 cells in the GVHD target tissues but expands IL-10-producing Tr1 cells, leading to effective prevention or treatment of GVHD. Administration of the anti-IL-2 antibody also preserves CD8⁺ memory T progenitors and effectors cells in the lymphoid tissues where they mediate GVL effect.

As demonstrated in the working examples, administration of tolerogenic anti-IL-2 mAb early after allo-HCT in mice markedly attenuates acute GVHD while preserving GVL activity that is dramatically stronger than observed with tacrolimus (TAC) treatment. In certain embodiments, the anti-IL-2 antibody is administered to the subject on the same day of receiving HCT, within 1 day of receiving HCT, within 2 days of receiving HCT, and within 3 days of receiving HCT. In certain embodiments, the anti-IL-2 antibody is administered to the subject receiving HCT before or immediately after the onset of GVHD. The anti-IL-2-treatment down-regulated activation of IL-2-Stat5 pathway and reduced production of GM-CSF. In GVHD target tissues, enhanced T cell PD-1 interaction with tissue-PD-L1 led to reduced activation of AKT-mTOR pathway and increased expression of Eomes and Blimp-1, increased T cell anergy/exhaustion, expansion of Foxp3⁺ Treg and Foxp3-IL-10-producing Tr1 cells, and depletion of GM-CSF-producing Th1/Tc1 cells. In recipient lymphoid tissues, lack of donor T cell PD-1 interaction with tissue-PD-L1 preserved donor PD-1⁺ TCF-1⁺Ly108⁺ CD8⁺ T memory progenitors (Tmp) and functional effectors that have strong GVL activity. The anti-IL-2 and TAC treatment have qualitatively distinct effects on donor T cells in the lymphoid tissues, and CD8⁺ Tmp cells are enriched with the anti-IL-2 treatment compared to TAC treatment. Thus, administration of tolerogenic anti-IL-2 mAb early after Allo-HCT represents a novel approach for preserving GVL activity while preventing acute GVHD.

Immunosuppressive medications such as TAC are routinely used to prevent GVHD in patients after Allo-HCT, but they can also inhibit GVL activity [34, 35]. Disclosed herein is a novel approach of preventing GVHD while preserving GVL activity by administration of tolerogenic anti-IL-2-JES6 early after HCT. Surprisingly, the anti-IL_2 JES6 antibody enables GVHD target tissue PD-L1 to effectively tolerize infiltrating T cells, leading to effective prevention of aGVHD, while preserving strong GVL activity that is much more effective than TAC under conditions where anti-IL-2 and TAC have similar effects on GVHD. Other anti-IL-2 antibodies such as anti-IL-2-S4B6 did not achieve the tolerogenic effects.

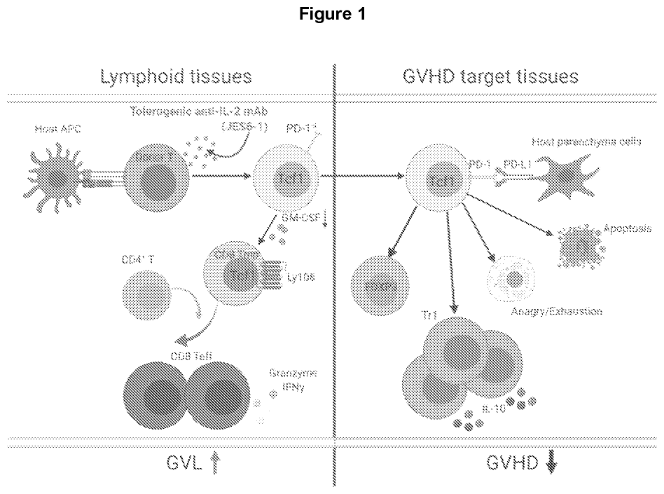

The mechanisms whereby tolerogenic anti-IL-2-JES6 treatment preserves GVL activity while preventing GVHD involve multiple steps is illustrated in FIG. 1. First, anti-IL-2-JES6 treatment inhibits activation of IL-2-Stat5 signaling pathway in the donor T cells and reduces their production of GM-CSF. Second, in the lymphoid tissues, due to relative lack of PD-L1-expression by recipient cells, the PD-1⁺ TCF-1⁺Ly108⁺ CD4⁺ and CD8⁺ Tmp cells are well preserved. Moreover, CD4⁺ T cells help the CD8⁺ Tmp differentiate into CD39⁺Ly108⁺ functional Teff cells to mediate persistent GVL activity. Third, in GVHD target tissues, donor T cell PD-1 interacts with host-tissue PD-L1, leading to down-regulated activation of the AKT-mTOR pathway and upregulated activation of anergy/exhaustion related nuclear factors, including Eomes and Blimp-1. Consequently, many donor T cells in GVHD target tissues become anergic, exhausted or apoptotic, with depletion of GM-CSF⁺ T cells; at the same time, some T cells differentiate into Foxp3⁺ Treg cells or Foxp3-IL-10-producing Tr1 cells, and anti-IL-2-JES6 may augment IL-2 binding to IL-2Rα on the regulatory T cells to augment their expansion. The regulatory T cells further suppress the pathogenic T cell function in the GVHD target tissues.

Tolerogenic anti-IL-2-JES6 treatment can affect both naïve and memory T cells in the graft. Conventional memory T cells express IL-2Rβ, and naïve T cells upregulate IL-2Rβ expression after activation [36]. Memory T cells in the graft have reduced GVHD capacity with preserved GVL activity in mice [37-39] and in patients [40]. Although not wishing to be bound by theory, administration of tolerogenic anti-IL-2 mAb may prevent GVHD while preserving GVL effects mediated by both naïve and memory T cells in the graft.

The experimental data disclosed herein provide new insights into how to separate GVHD from GVL activity mediated by the same alloreactive donor T cell population. Anti-IL-2-JES6-treatment reduced IL-2-Stat5 activation independent of host tissue PD-L1, but the treatment upregulated T cell expression of PD-1 and reduced activation of AKT-mTOR pathways in the T cells from GVHD target tissues in the host-tissue PD-L1-dependent manner. Therefore, simultaneous reduction of activation by blocking IL-2 effect and augmentation of inhibition by PD-1 signaling enable inhibition of AKT-mTOR pathway in the T cells in GVHD target tissues, leading to prevention of GVHD; lack of PD-1 interaction with host tissue PD-L1 in the lymphoid tissues allows better alloreactive T cell survival, leading to stronger GVL effect.

As demonstrated in the working examples, PD-L1/PD-1 interaction augments differentiation and expansion of Foxp3-IL-10-producing Tr1 cells. IL-10⁺ Tr1 cells represent the major regulatory T cell population in allo-HCT recipients; moreover, Eomes is required for donor T cell differentiation into FoxP3-IL-10⁺ Tr1 cells, and Blimp-1 augments expansion of Tr1 cells [17]. The observations that anti-IL-2-JES6 treatment upregulated donor CD4⁺ T expression of Eomes in both WT and PD-L1^{-/-} recipients, but upregulated expression of Blimp-1 only in WT but not in PD-L1^{-/-} recipients suggest that reduction of AKT-mTOR activation by blocking IL-2 effect alone is able to upregulate Eomes in the absence of PD-1 signaling; but simultaneous reduction of AKT-mTOR activation by blocking IL-2 effect and inhibition of AKT-mTOR activation by PD-1 signaling triggered by tissue PD-L1 is required to upregulate expression of Blimp-1. Therefore, anti-IL-2-JES6 enables tissue PD-L1 to mediate differentiation and expansion of Tr1 cells in GVHD target tissues.

Anti-IL-2-JES6 treatment exploits differences in expression of PD-L1 by recipient GVHD target and lymphoid tissues that affect the ability of PD1⁺ TCF-1⁺Ly108⁺ CD8⁺ Tmp cells to cause GVHD and mediate GVL activity. Blockade of PD-1 interaction with PD-L1 can revive the function of TCF-1⁺ Tmp and Teff cells [33]. The paucity of PD-L1-expressing cells in recipient lymphoid tissues preserves donor-type PD-1⁺ TCF-1⁺Ly108⁺ CD8⁺ Tmp and their derivatives locally where they mediate GVL activity. In contrast, the abundance of PD-L1-expressing cells in the GVHD target tissues such as colon and liver tolerizes donor-type PD-1⁺ TCF-1⁺Ly108⁺ Tmp and their derivative Teff cells locally, thereby preventing GVHD. Thus, anti-IL-2-JES6 treatment allows donor-type PD-1⁺ TCF-1⁺Ly108⁺ CD8⁺ Tmp cells to mediate GVL activity in lympho-hematopoietic compartment without causing aGVHD in parenchymal tissues, even though CD8⁺ Tmp cells can mediate persistence of GVHD [24].

From the foregoing, it will be appreciated that specific embodiments of the invention have been described herein for purposes of illustration, but that various modifications may be made without deviating from the scope of the invention. The examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

### Example 1. Materials and Methods

Induction and assessment of GVHD, measurement of cytokines in serum, flow cytometry analysis and sorting, histopathology and histoimmunofluorescent staining, single cell RNA sequencing library construction using the 10× genomics chromium platform, and statistical analysis are described in previous publications [13] and detailed below.

Mice: BALB/c (H-2^{d}) and C57BL/6 (H-2^{b}) mice were purchased from National Cancer Institute (Frederick, MD). IL-10^{-/-} C57BL/6 mice (H-2^{b}) were purchased from the Jackson Laboratory. PD-L1^{-/-} BALB/c breeders were provided by Dr. L. Chen (Yale University, New Haven). All mice were maintained in a pathogen-free room in City of Hope Animal Research Center. All experiments were approved by IACUC at City of Hope.

Induction and assessment of GVHD: BALB/c recipients were exposed to 850 cGy total body irradiation (TBI) with the use of a [137Cs] source 8 hours before HCT, and then given C57BL/6 donor spleen cells (2.5×10⁶-5.0×10⁶), Thy1.2⁺ cells (1×10⁶) and T cell-depleted BM (TCD-BM) cells (2.5×10⁶) by tail vein injection. C57BL/6 recipients were exposed to 1300 cGy total body irradiation (TBI) with the use of a [137Cs] source 8 hours before HCT, and then given A/J donor spleen cells (2.5×10⁶) and T cell-depleted BM (TCD-BM) cells (2.5×10⁶) by tail vein injection. The bone marrow was depleted of T cells by using biotin-conjugated anti-CD4 and anti-CD8 mAbs, and streptavidin Microbeads (Miltenyi Biotec, Germany), followed by passage through an autoMACS Pro cell sorter (Miltenyi Biotec, Germany). For GVL experiments, Luc⁺ B-cell leukemia/lymphoma 1 (BCL1) cells (5-10×10⁶) were injected intraperitoneally at the same time when donor bone marrow and spleen cells were injected intravenously. GFP+ blast crisis chronic myeloid leukemia (BC-CML) cells (1×10⁶) were injected intravenously at the same time when donor bone marrow and spleen cells were injected. In vivo imaging of tumor growth was monitored by using Lago IVIS100 charge-coupled device imaging system. The assessment and scoring of clinical signs of acute GVHD has been described previously [42].

Cell lines: Luciference transfected BCL-1 cell line was provided by Dr. Christopher Contag at Stanford University (Stanford, CA). GFP+ blast crisis chronic myeloid leukemia (BC-CML) cell line was provided by Dr. Warren Shlomchik at Pittsburgh University (Pittsburgh, PA).

Antibodies and FACS analysis: Anti-IL-2 mAb (JES6-1A12, that is, JES6-1), anti-IL-2 mAb (S4B6) and anti-NK1.1 (PK136) for in vivo treatment was purchased from Bio X Cell (West Lebanon, NH). ChromPure Rat IgG (012-000-003) was purchased from Jackson ImmunoResearch Laboratories, Inc (West Grove, PA, USA). mAbs specific for MHCII (M5/114.15.2) and T-bet (4B10) were purchased from BD Bioscience. mAbs specific for TCRβ (H57-597), H-2Kb (AF6-88.5), CD4 (RM4-5), CD8a (53-6.7), CD11b (M1/70), CD11c (N418), Ly6G (RB6-8C5), Ly6C (HK1.4), pMTOR (MRRBY), CD39 (24DMS1) were purchased from ThermoFisher Bioscience. mAbs specific for CD24 (M1/69), Pro-IL-13 (NJTEN3), B7H1 (H1M5), pAKT473 (SDRNR), KLRG1 (2F1/KLRG1), CD107a (1D4B), GranzymeB (QA16A02), ly108 (330-AJ), TIM3 (RMT3-23), IFN-γ (XMG1.2), TNF-α (MP6-XT22), Eomes (Dan 11 mag), GM-CSF (MP1-22E9), IL-10 (JES5-16E3), Blimp-1 (5E7), perforin (eBioOMAK-D), CD127 (SB/199), IL-2 (JES5-5H4), Foxp3 (FJK-16s), NKp46 (29A1.4) and mouse Breg staining kit (anti-mouse CD19/CD5/CD1d) were purchased from Biolegend (San Diego, CA). CeIIROX™ Green Reagent (for detection of oxidative stress), anti-mouse CD3e (145-2C11), anti-mouse CD19 (eBio1D3) and anti-mouse NK1.1 (PK136) was purchased from ThermoFisher Scientific. Flow cytometry analyses were performed with an Attune NxT Cytometer (ThermoFisher Scientific) and BD LSRFortessa (Franklin Lakes, NJ), and the resulting data were analyzed with FlowJo software V10 (Tree Star, Ashland, OR).

Isolation of cells from spleen, mesenteric lymph node, liver and large Intestine: Spleen, lymph node and liver tissue were mashed through a 70 μm cell strainer, and MNC were isolated from the cell suspensions with percoll. Intestine was cut first longitudinally and then laterally into pieces of approximately 0.5 cm length. Tissue pieces were incubated with 20 mL of predigestion solution (1×HBSS without containing 5 mM EDTA, 5% fetal bovine serum (FBS), 1 mM DTT) for 20 minutes at 37° C. under continuous shaking, then passed through 100 μm strainer and held for at least 10 minutes on ice. Intestine epithelia lymphoid cells in the supernatant were collected. Then tissue pieces were digested with enzyme to isolate the lamina propria cells, following the protocol of Lamina Propria Dissociation Kit (Miltenyi Biotec).

Histopathology: Tissue specimens were fixed in formalin before embedding in paraffin blocks, sectioned and stained with H&E. Slides were examined at 100× (liver) or 200× (small intestine and colon) magnification and visualized with Zeiss Observer II. Tissue damage was blindly assessed according to a defined scoring system, as described previously [42]. Liver GVHD was scored by the severity of lymphocytic infiltrate, number of involved tracts and severity of liver cell necrosis, the maximum score is 9. Gut GVHD was scored by mononuclear cell infiltration and morphological aberrations (e.g. hyperplasia and crypt loss), with a maximum score of 8.

Bioluminescent imaging: Mice were given with luciferase⁺ BCL1 cells (BCL1/Luc⁺) by i.p. injection. For in vivo imaging of tumor growth, 200 μl firefly luciferin was injected i.p. (Caliper Life Sciences, Hopkinton, MA), and mice were anesthetized for analysis of tumor cell burden by using an IVIS100 (Xenogen) and AmiX (Spectral) imaging system. Data were analyzed by using Amiview software purchased from Spectral Instruments Imaging (New York, NY).

In vivo BrdU labeling: T cell proliferation was measured with a single i.p. injection of BrdU (2.5 mg/mouse, 100 mg/g) 3 hours before tissue harvesting. Analysis of donor CD8⁺ T cells for BrdU incorporation was performed according to the manufacturer's instructions (BD Pharmingen).

mRNA sequencing library preparation and sequencing: RNA concentration was measured by NanoDrop 1000 (Thermo Fisher Scientific, Waltham Massachusetts, US), and RNA integrity was determined using Bioanalyzer (Agilent). Library construction of 280 ng total RNA for each sample was made by using KAPA Stranded mRNA-Seq Kit (Illumina Platforms) (Kapa Biosystems, Wilmington, USA) with 10 cycles of PCR amplification. Libraries were purified using AxyPrep Mag PCR Clean-up kit (Thermo Fisher Scientific). Each library was quantified using a Qubit fluorometer (Life Technologies), and the size distribution was assessed using the 2100 Bioanalyzer (Agilent Technologies, Santa Clara, USA). Sequencing was performed on an Illumina® Hiseq 2500 (Illumina, San Diego, CA, USA) instrument using the TruSeq SR Cluster Kit V4-cBot-HS (Illumina®) to generate 51 bp single-end reads sequencing with v4 chemistry. Quality control of RNA-Seq reads was performed using FastQC. Bioconductor package “clusterProfiler” v3.10.1 was used for GSEA analysis to generate the NES and P value, while “enrichplot” v1.2.0 was used to generate the GSEA plot [43].

Single-cell sequencing library construction using the 10× Genomics Chromium Platform: Library preparation was done with the Chromium Single Cell 5′ Reagent Kits from 10× Genomics according to manufacturer's protocol. Cellular suspensions were loaded on a Chromium Controller instrument (10× Genomics) to generate single-cell gel bead-in-emulsions (GEMs). GEM-reverse transcriptions (GEM-RTs) were performed in a Veriti 96-well thermal cycler (Thermo Fisher Scientific). After reverse transcription, GEMs were harvested, and the cDNAs were amplified and cleaned with the SPRIselect Reagent Kit (Beckman Coulter). Indexed sequencing libraries were constructed using the Chromium Single-Cell 5′ Library Kit (10× Genomics) for enzymatic fragmentation, end-repair, A-tailing, adaptor ligation, ligation cleanup, sample index PCR, and PCR cleanup. The purity and library size were validated by capillary electrophoresis using 2,100 Bioanalyzer (Agilent Technologies). The quantity was measured fluorometrically using Qubit dsDNA HS Assay Kit from Invitrogen.

Libraries were sequenced with a NovaSeq 6000 instrument (Illumina) to a depth of 35 k-40 k reads per cell. Raw sequencing data were processed using the 10× Genomics' Cell Ranger pipeline (version 3.1.0) to generate FASTQ files and aligned to mm 10 genome to generate gene expression counts. The subsequent data analysis was performed using “Seurat v3.0” package and R scripts. Cells with mitochondrial read >10% and <200 detectable genes were considered as low-quality and filtered out. Normalized and scaled data were clustered using the top significant principal components of 2000 highly variable genes and resolution of 0.4 using “Seurat”. The t-distributed stochastic neighbor embedding (t-SNE) algorithm was used to visualize the resulting clusters. Cluster specific markers were identified using “Seurat” to generate the heatmap of marker genes in these cell clusters. Genes were compared between different clusters using Bioconductor package “Limma” and log 2 normalized data. Gene Set Enrichment analysis (GSEA) v3 was performed using genes ranked by the −log 10 of “Limma” comparison P value to evaluate the significant activation or inhibition of the Hallmark gene sets in MSigDb (www.gsea-msigdb.org/gsea/msigdb/genesets.jsp?collection=H). All plots were generated using either “Seurat” or “ggplot2” package in R.

Statistical analysis: Data were displayed as mean±SEM. Mortality rates in different groups were compared by log-rank test. Comparison of body weight in different groups was analyzed by nonlinear regression (curve fit). Comparison of means for more than two groups was analyzed by 1-way ANOVA or 2-way ANOVA multiple comparisons, while comparison of two means was analyzed by unpaired two-tailed Student t-test (Prism, version 8.0; GraphPad Software), P less than 0.05 was considered as statistically significant (*p<0.05, **p<0.01, ***p<0.001, ****p<0.0001).

### Example 2. Administration of Tolerogenic Anti-IL-2-JES6 mAb Prevents aGVHD and Preserves GVL Activity More Effectively than Tacrolimus

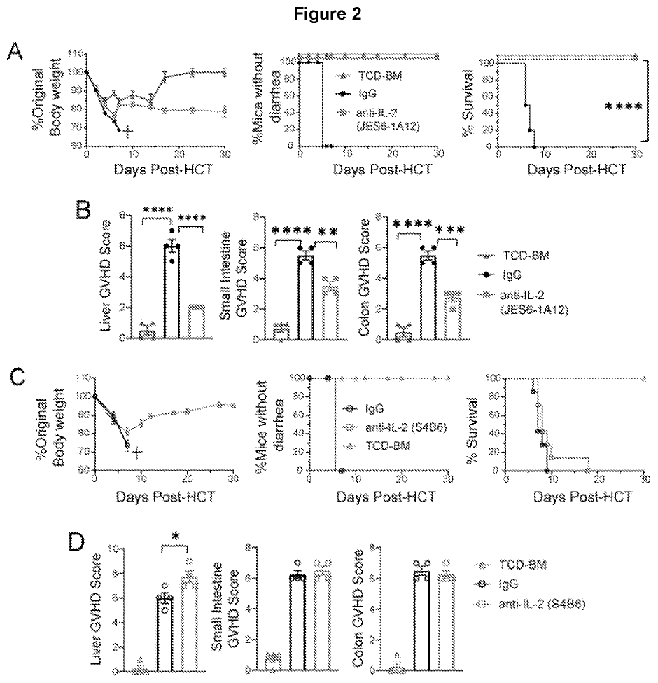

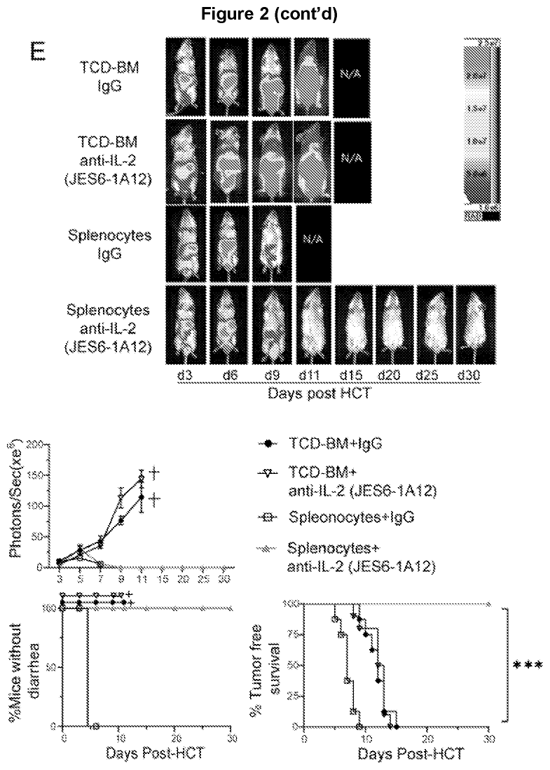

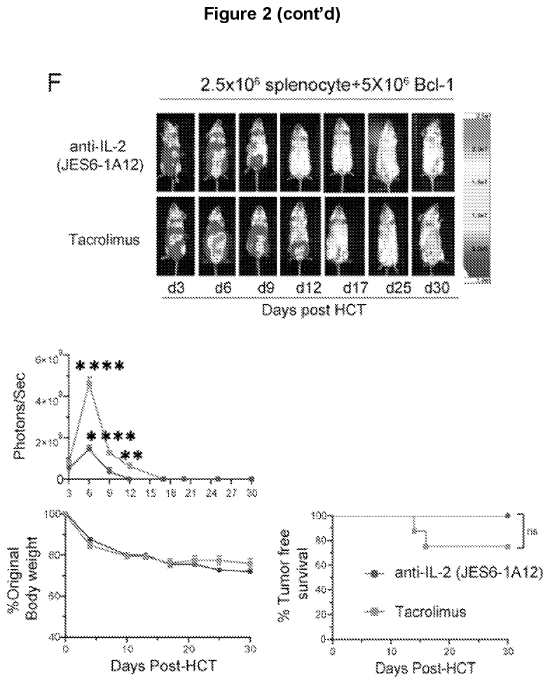

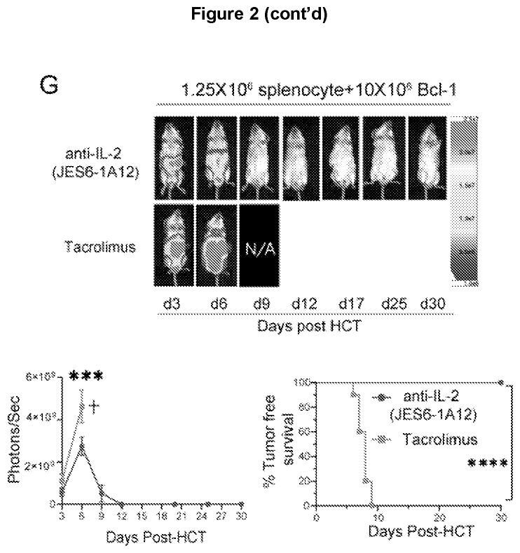

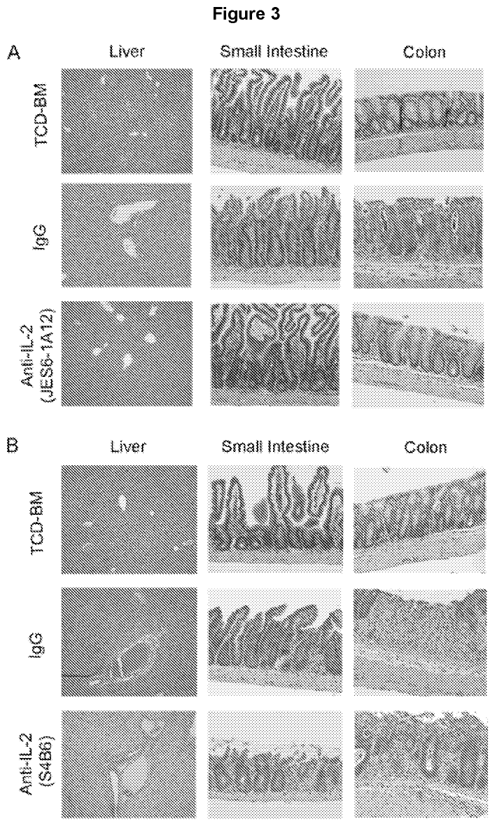

It was proposed that IL-2 from donor CD4⁺ T cells may make alloreactive donor T cells resistant to induction of tolerance (i.e. anergy, exhaustion and apoptosis) by host-tissue PD-L1 [13]. Thus, it was tested whether administration of tolerogenic anti-IL-2-JES6 (JES6-1A12) mAb that block IL-2 interaction with IL-2Rβ on conventional Tcells [27] could prevent GVHD and preserve GVL activity. Accordingly, irradiated BALB/c recipients were engrafted with splenocytes (5×10⁶) and TCD-BM cells (2.5×10⁶) from MHC-mismatched C57BL/6 donors. Recipients given TCD-BM alone were used as GVHD-free controls. Recipients were treated with rat anti-IL-2-JES6 or control rat IgG at a dose of 500 μg/mouse i.p. on days 0, 2, 4 and 6 after HCT. As compared to IgG treatment, anti-IL-2-JES6 treatment limited loss of body weight and completely prevented diarrhea, and all recipients survived for more than 30 days. Moreover, anti-IL-2-JES6 treatment prevented GVHD target tissues (liver, small intestine and colon) damage (FIGS. 2A-2B and 3A). In contrast, administration of non-tolerogenic anti-IL-2-S4B6 that augment IL-2 interaction with IL-2Rβ on conventional T cells did not prevent acute GVHD (FIGS. 2C-2D and 3B). These results indicate that administration of tolerogenic anti-IL-2-JES6 but not non-tolerogenic anti-IL-2-S4B6 greatly attenuated the severity of aGVHD.

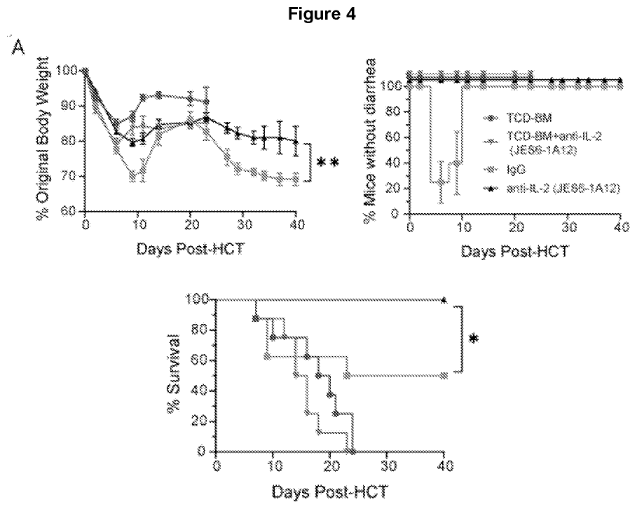

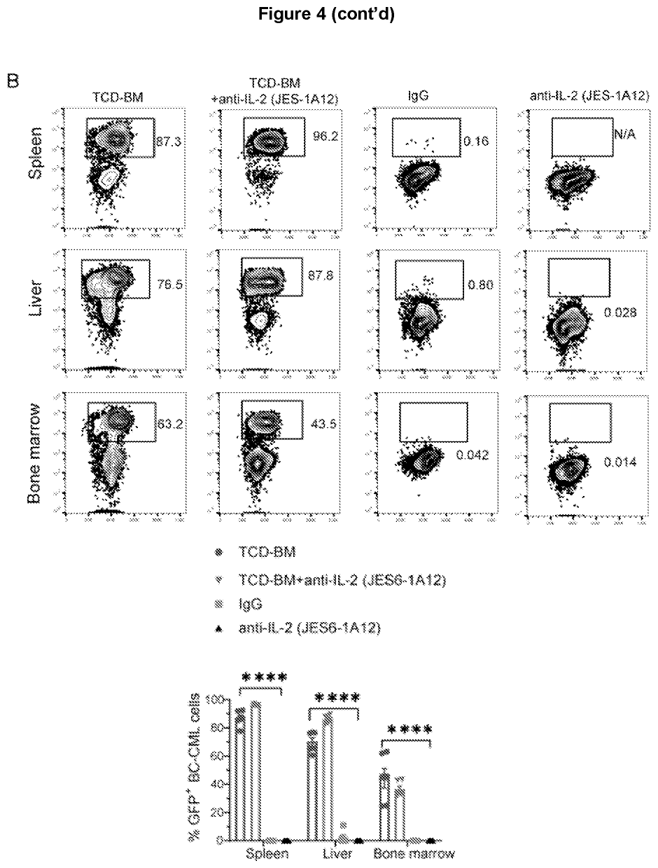

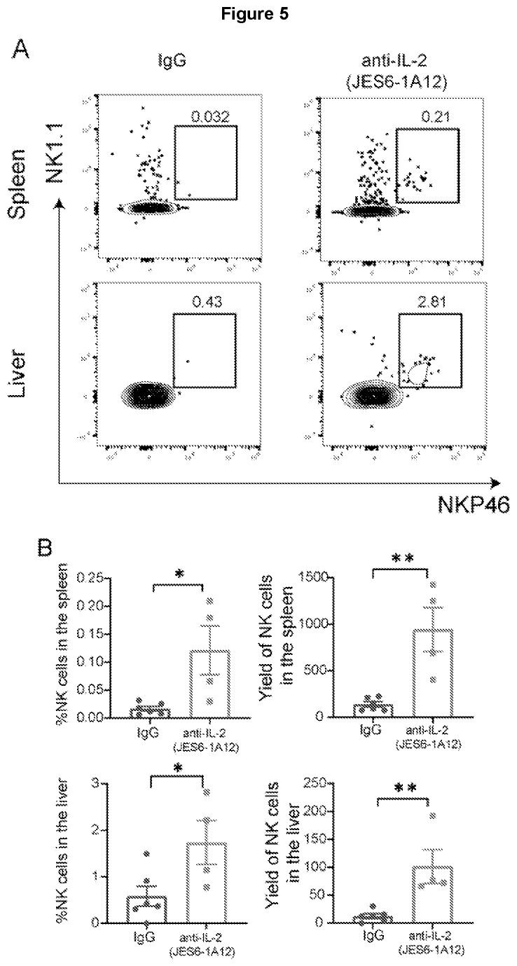

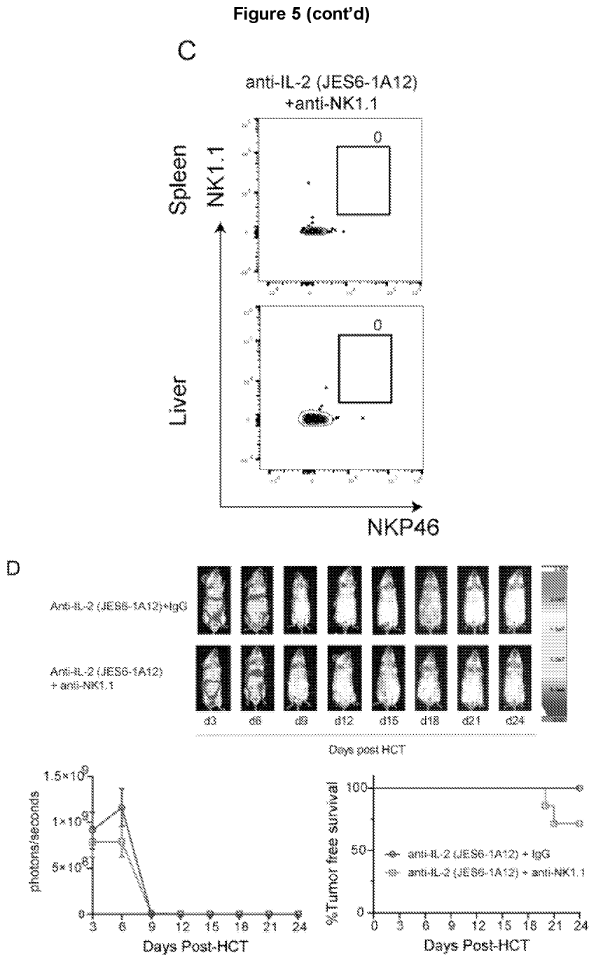

To assess the effect of tolerogenic anti-IL-2-JES6 treatment on GVL activity, BALB/c recipients were inoculated with luciferase-transfected BCL1 cells (BCL1/Luc, 5×10⁶/mouse, i.p.) before HCT. BCL1/Luc cell growth was monitored by in vivo bioluminescent imaging (BLI). BCL1/Luc tumor cells grew rapidly in recipients engrafted with TCD-BM; anti-IL-2-JES6-treatment did not have significant impact on the tumor growth; and all recipients died within 20 days after HCT (FIG. 2E). BCL1/Luc tumor cells grew transiently in IgG-treated recipients engrafted with splenocytes and TCD-BM cells, but all recipients died with GVHD within 10 days after HCT. BCL1/Luc tumor cells also grew transiently in anti-IL-2-JES6-treated recipients and were eliminated by day 11, but all mice survived for more than 30 days without tumor relapse (FIG. 2E). Treatment with anti-IL-2-JES6 also eliminated GVL-resistant BC-CML tumor cells [28], while preventing aGVHD (FIG. 4). Although anti-IL-2-JES6 treatment augmented NK cell expansion, depletion of NK cells had no impact on GVL activity (FIG. 5). Taken together, these results show that treatment with tolerogenic anti-IL-2-JES6 effectively controlled GVHD while preserving strong GVL activity mediated by alloreactive T cells.

The calcineurin inhibitor tacrolimus (TAC) is widely used clinically to prevent aGVHD, in part by inhibiting endogenous IL-2 production in alloactivated donor T cells. Therefore, it was of interest to compare the effects of anti-IL-2-JES6 and tacrolimus regarding their respective abilities to prevent aGVHD while preserving GVL activity. BALB/c recipients engrafted with spleen (2.5×10⁶) and TCD-BM (2.5×10⁶) cells from C57BL/6 donors and challenged with 5×10⁶ BCL1/Luc cells on day 0 were treated with anti-IL-2 on days 0, 2, 4, and 6 after HCT or with i.p. injection of TAC (0.75 mg/kg) daily for up to 21 days. The two groups showed similar loss of body weight, and survival was not statistically different between the 2 groups during the first 30 days after HCT. Both groups cleared tumor cells by 12-17 days after HCT (FIG. 2F).

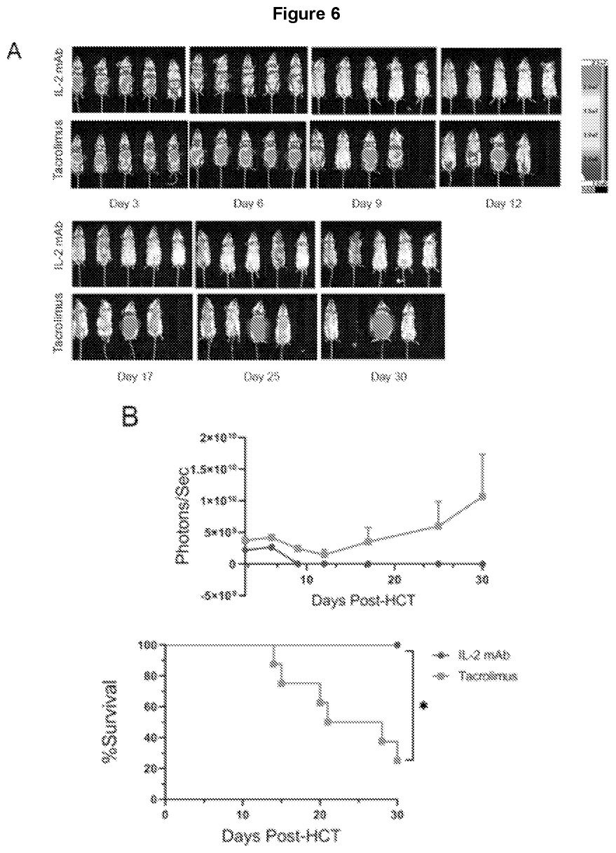

In further experiments, BALB/c recipients engrafted with C57BL/6 BM cells and a lower number of spleen cells (1.25×10⁶) were challenged with i.p. inoculation of 5 or 10×10⁶ Luc/BCL1 cells, with the same regimen of anti-IL-2-JES6 or TAC. In recipients challenged with 5×10⁶ BCL1/Luc cells, tumor cells disappeared before day 12 in all anti-IL-2-JES6 treated, but only 60% of TAC-treated recipients cleared tumor by day 17, while the other 40% died with progressive tumor growth by 30 days after HCT (FIG. 6). In recipients challenged with 10×10⁶ BCL1/Luc cells, all anti-IL-2-JES6-treated recipients cleared tumor cells by day 12 after HCT, but all TAC-treated recipients died with progressive tumor growth by 9 days after HCT (FIG. 2G). Thus, under the conditions used for these experiments, anti-IL-2-JES6 and TAC had comparable ability to prevent acute GVHD, but anti-IL-2-JES6 treatment preserved GVL activity, while TAC did not.

### Example 3. Prevention of aGVHD by Tolerogenic Anti-IL-2-JES6 Requires PD-L1 Expression by GVHD Target Tissues

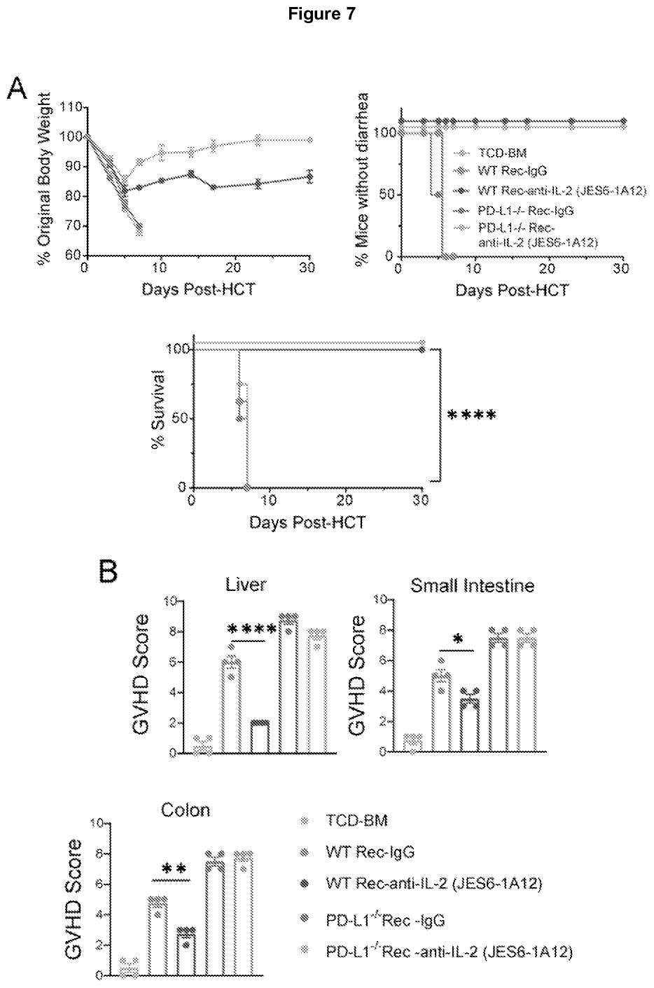

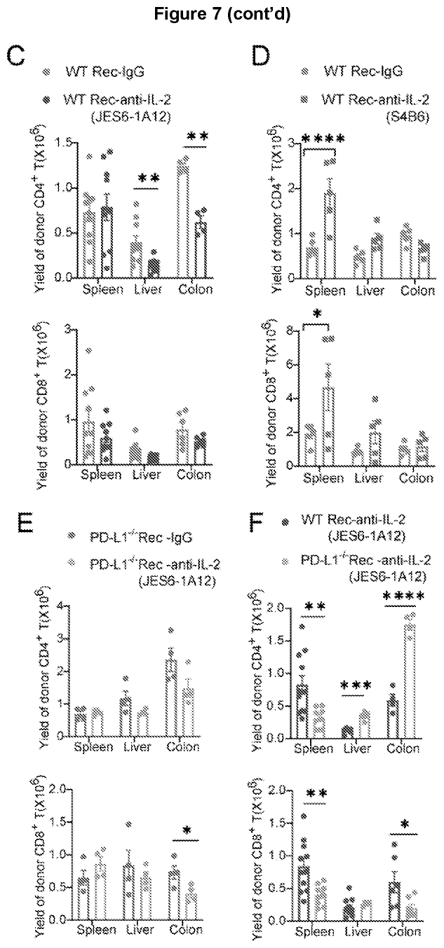

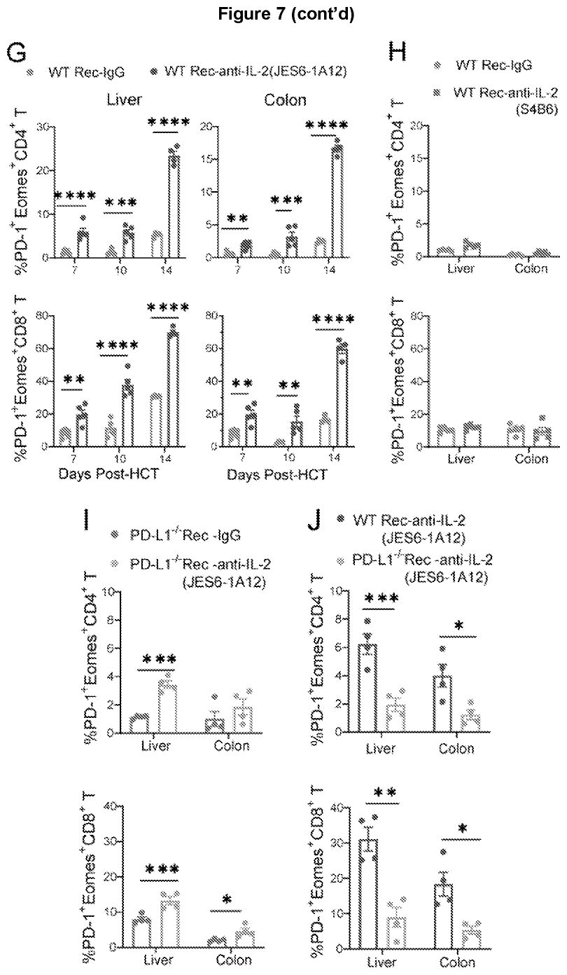

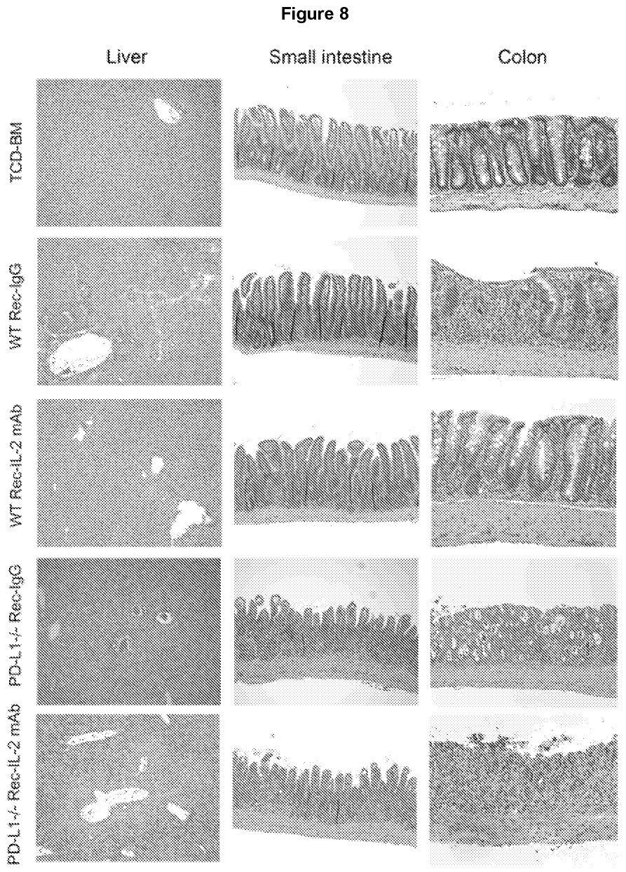

Prevention of GVHD by depleting donor CD4⁺ T cells that produced IL-2 was host-tissue PD-L1-dependent [13]. Therefore, it was tested whether prevention of GVHD by tolerogenic anti-IL-2-JES6 also depends on recipient PD-L1. Anti-IL-2-JES6 attenuated the severity of GVHD in WT recipients but not in PD-L1^{-/-} recipients (FIGS. 7A, 7B and 8). Anti-IL-2-JES6 treatment markedly ameliorated tissue damage in the liver, small intestine and colon of WT recipients as compared with IgG-treated recipients. In contrast, anti-IL-2-JES6 treatment did not ameliorate tissue damage at all in PD-L1^{-/-} recipients (FIGS. 7B and 8). As compared to IgG treatment, anti-IL-2-JES6-treatment reduced the yield of donor CD4⁺ T cells although not CD8⁺ T cells in the liver and colon at day 7 in WT recipients (FIG. 7C). In contrast, anti-IL-2-S4B6-treatment expanded donor-type CD4⁺ and CD8⁺ T cells in the spleen but produced no significant changes in the liver or colon (FIG. 7D). As compared to IgG treatment, the effect of anti-IL-2-JES6 treatment was not apparent PD-L1^{-/-} recipients (FIG. 7E). But as compared with WT recipients, the yield of CD4⁺ T cells in the liver and colon at day 7 was higher in PD-L1^{-/-} recipients (FIG. 7F). These results also indicate that tolerogenic anti-IL-2-JES6 augments host-tissue PD-L1-medated protection against aGVHD.

Upregulation of expression of PD-1 and Eomes is a feature of anergic/exhausted T cells [29]. Anti-IL-2-JES6-treatment markedly increased the percentages of PD-1⁺Eomes⁺ CD4⁺ and CD8⁺ T cells in the GVHD target tissues liver and colon in a time-dependent manner in the WT recipients (FIG. 7G), while anti-IL-2-S4B6 did not have these effects (FIG. 7H). As compared with IgG treatment, anti-IL-2-JES6 treatment also increased the percentage of PD-1⁺Eomes⁺ CD4⁺ and CD8⁺ T cells in the liver and colon tissues at day 6 after HCT in the PD-L1^{-/-} recipients (FIG. 7I). Later time points were not available for evaluation, due to death of most PD-L1^{-/-} recipients by 7 days after HCT. The percentages of PD-1⁺Eomes⁺ CD4⁺ and CD8⁺ T cells in the liver and colon tissues of anti-IL-2-JES6 treated PD-L1^{-/-} recipients were markedly lower than in anti-IL-2-JES6-treated WT recipients at day 6 after HCT (FIG. 7J). These results indicate that tolerogenic anti-IL-2-JES6 treatment and tissue PD-L1 interaction with PD-1 synergistically augment T cell anergy/exhaustion of tissue infiltrating T cells, leading to prevention of aGVHD.

### Example 4. Prevention of GVHD by Tolerogenic Anti-IL-2-JES6 is Associated with Tissue PD-L1-Dependent Depletion of GM-CSF-Producing Th1 and Tc1 Cells

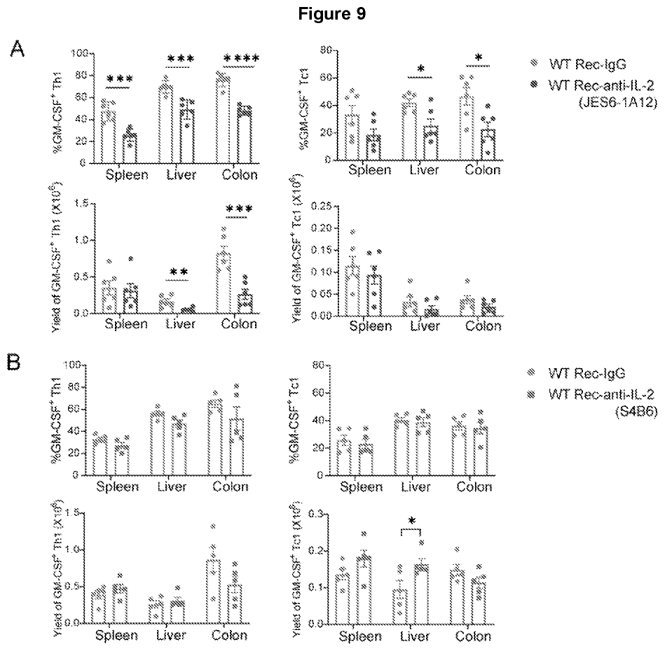

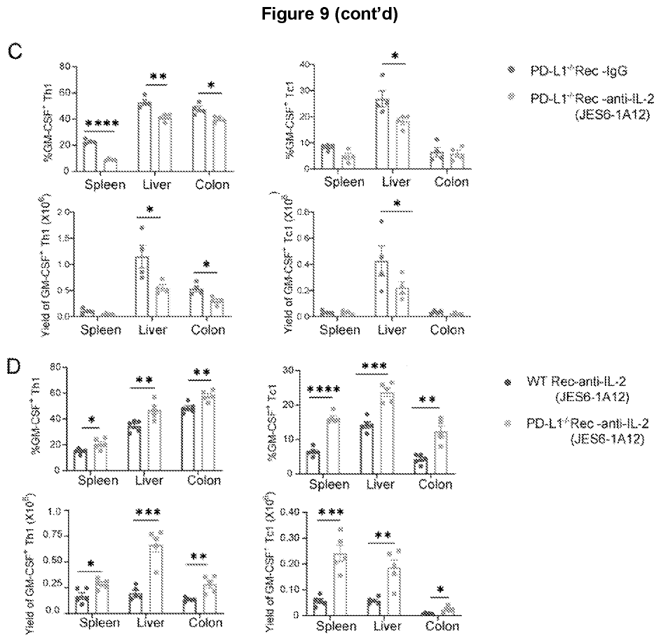

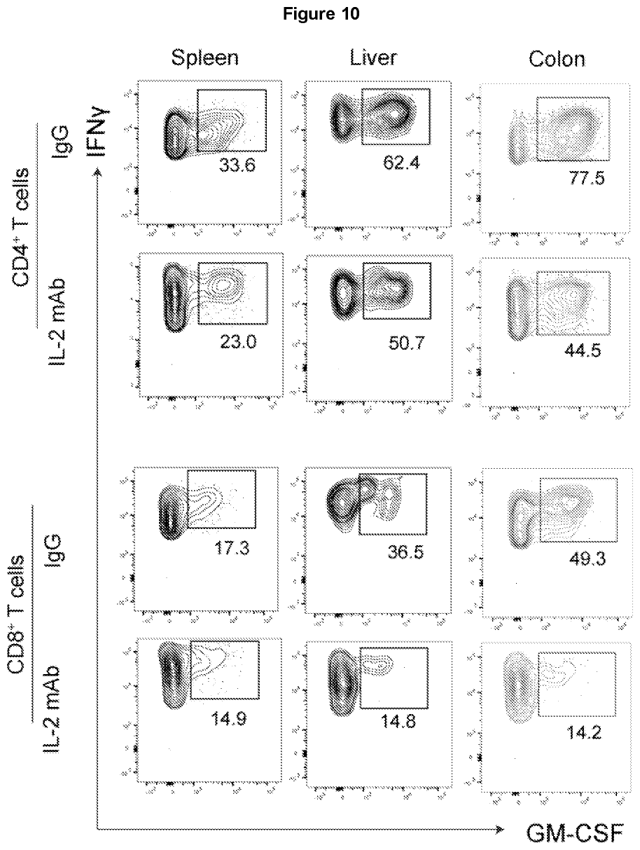

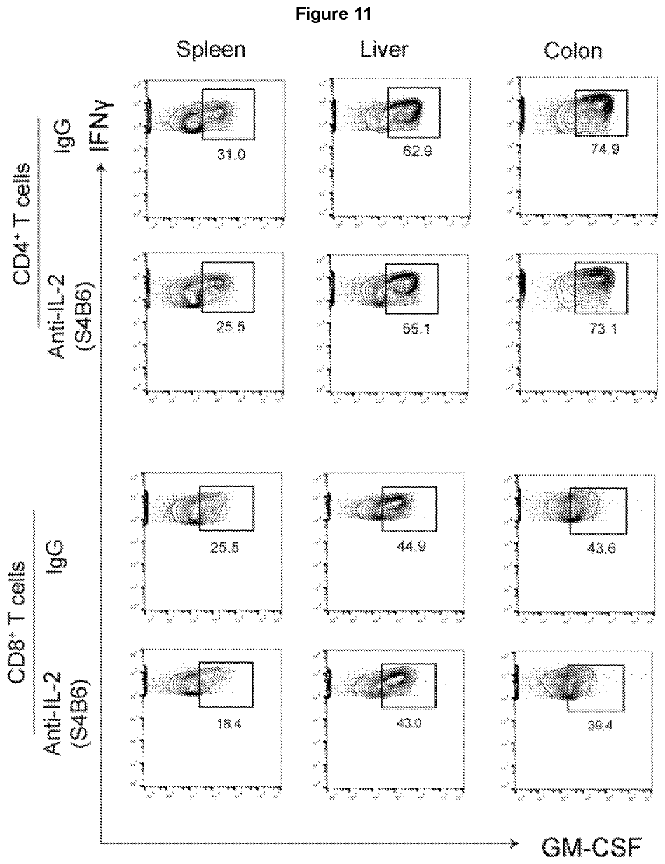

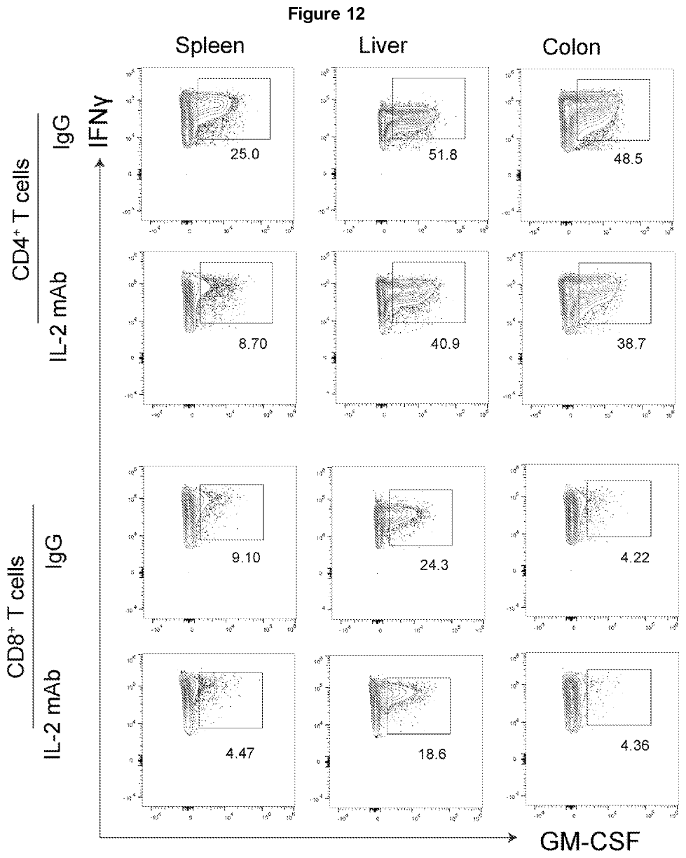

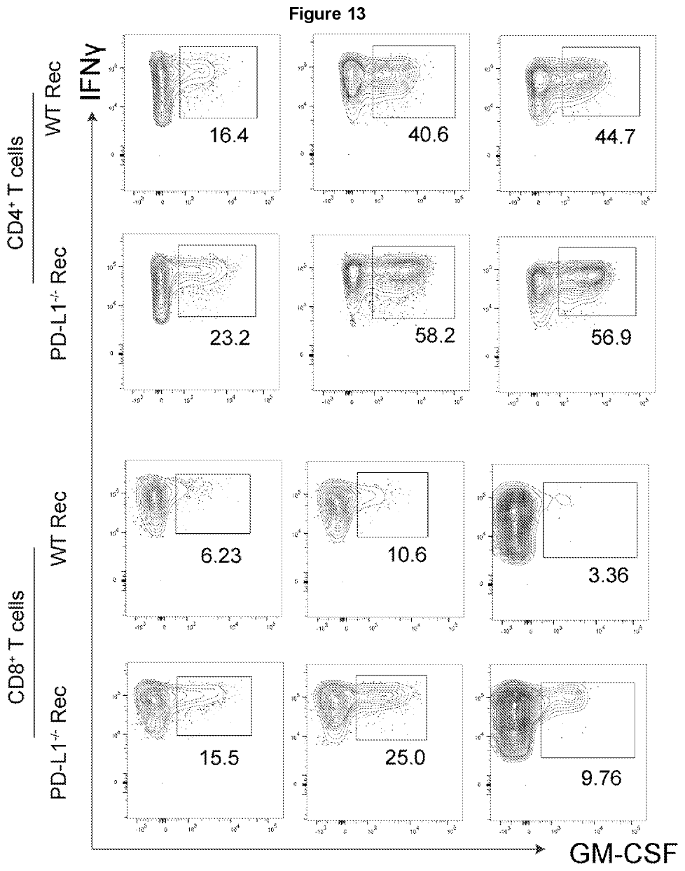

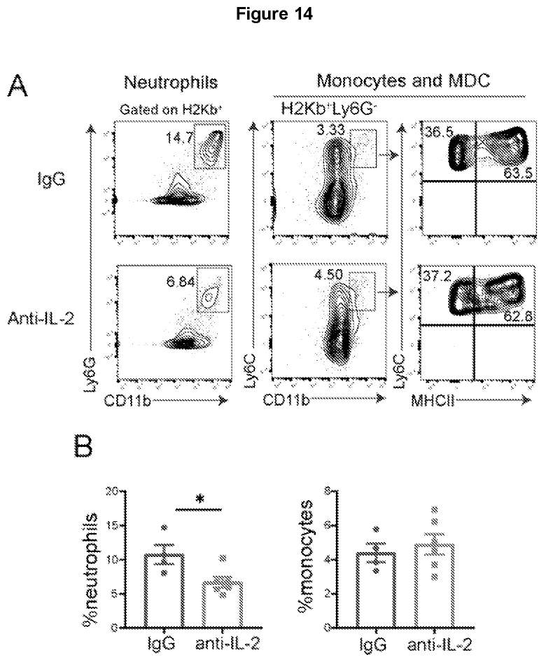

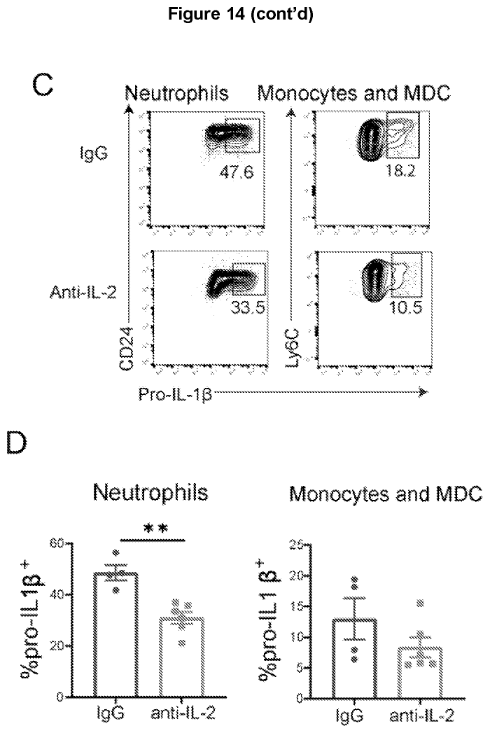

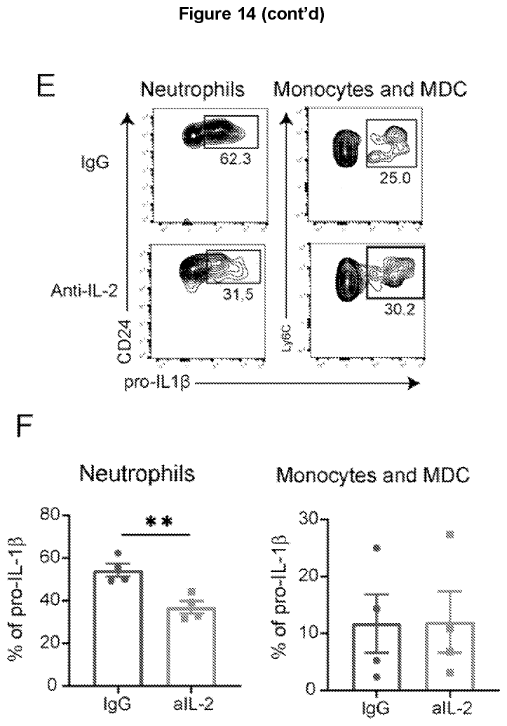

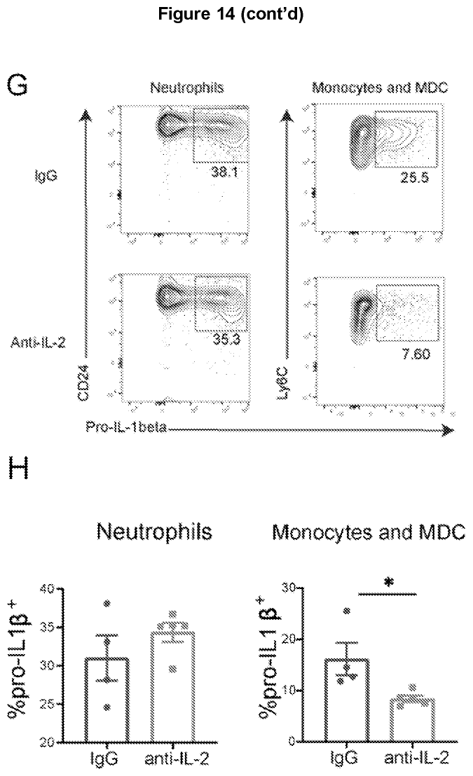

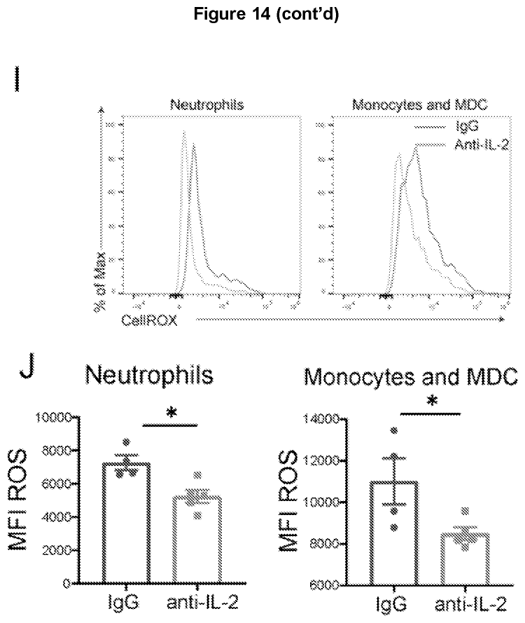

GM-CSF-producing Th1 and Tc1 cells play an essential role in aGVHD pathogenesis [30, 31]. GM-CSF-producing (GM-CSF⁺) Th1 and Tc1 cells were evaluated at day 6 after HCT. As compared with IgG treatment, anti-IL-2-JES6 treatment reduced the percentages and yield of GM-CSF⁺IFN-γ+Th1 in the liver and colon of WT recipients and reduced the percentages but not the yields of Tc1 in those issues (FIGS. 9A and 10), while anti-IL-2-S4B6 did not have these effects (FIGS. 9B and 11). As compared to IgG treatment, anti-IL-2-JES6 treatment also reduced the percentage of GM-CSF⁺ Th1 and Tc1 cells in the liver tissues although not in the spleen or colon tissues in the PD-L1^{-/-} recipients (FIGS. 9C and 12). The percentage and yield of GM-CSF⁺ Th1 and Tc1 cells in the spleen, liver and colon of anti-IL-2-JES6-treated PD-L1^{-/-} recipients were markedly higher than in anti-IL-2-JES6-treated WT recipients (FIGS. 9D and 13). The lower percentages of Th1 and Tc1 cells expressing GM-CSF induced by anti-IL-2-JES6 treatment in WT recipients were associated with lower infiltration of neutrophils and monocytes in the liver and colon and with their lower production of pro-IL-1p and ROS (FIG. 14). These results indicate that administration of tolerogenic anti-IL-2-JES6 and tissue PD-L1 interaction with PD-1 on T cells synergistically augment depletion of GM-CSF-producing Th1 and Tc1 cells in GVHD target tissues.

### Example 5. Prevention of aGVHD by Anti-IL-2-JES6 Requires PD-L1-Dependent Expansion of Tr1 Cells

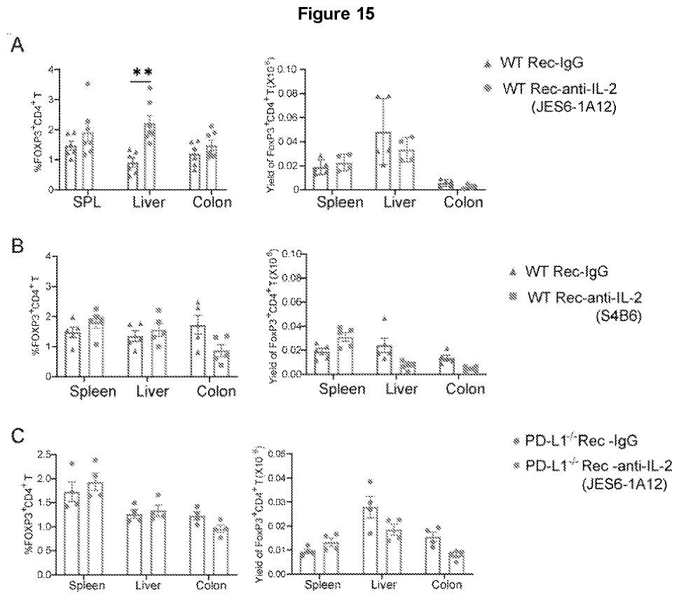

Anti-IL-2-JES6 but not anti-IL-2-S4B6 increased the percentage of Foxp3⁺ Treg cells in the liver of WT recipients (FIGS. 15A-15B). Neither anti-IL-2 had effect on the percentage or yield of Foxp3⁺ Treg cells in the colon (FIGS. 15A-15B). Anti-IL-2-JES6 treatment did not have a significant effect on Treg expansion in PD-L1^{-/-} recipients (FIG. 15C).

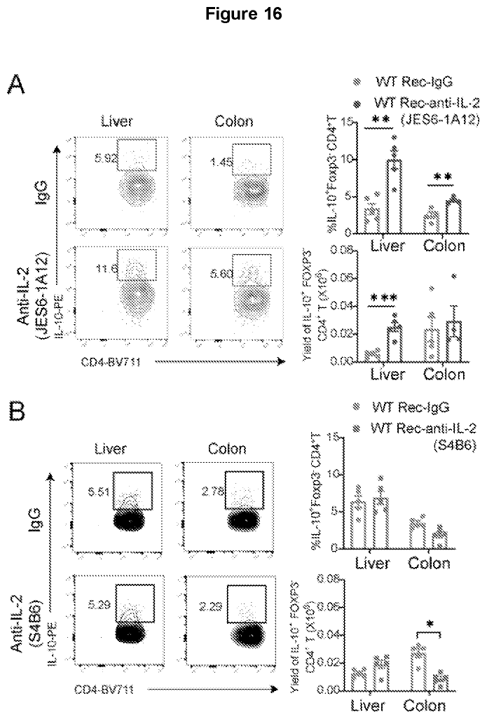

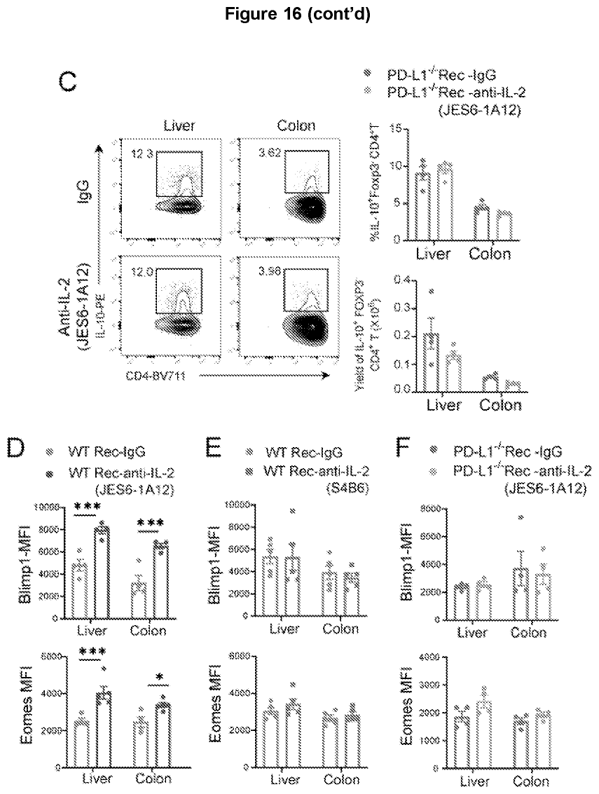

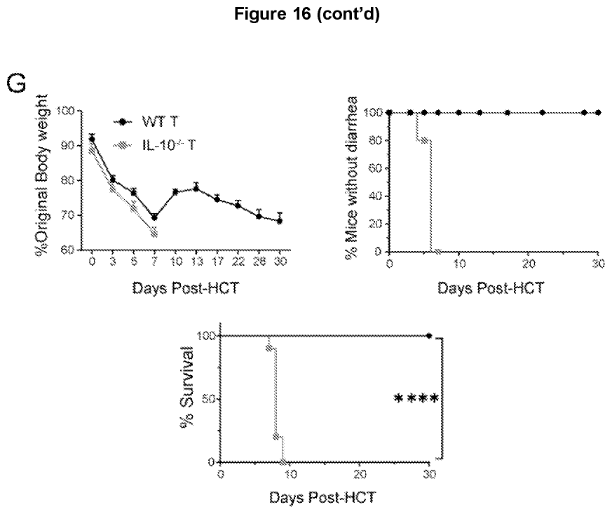

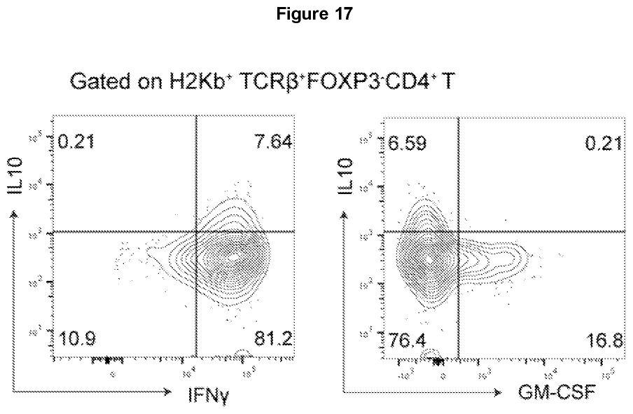

Foxp3-IL-10⁺ Tr1 cells represent the majority of regulatory T cells in allo-HCT recipients, and Tr1 cell expression of Eomes and Blimp-1 are required for Tr1 cell differentiation [17]. Since tissue infiltrating CD4⁺ T cells expressed higher levels of Eomes (FIG. 7G), the impact of anti-IL-2-JES6 treatment on Foxp3-IL-10⁺ Tr1 cell expansion at day 6 after HCT was evaluated. As compared with IgG treatment, anti-IL-2-JES6, but not anti-IL-2-S4B6, significantly increased the percentage of IL-10⁺ Tr1 cells among donor-type CD4⁺ T cells in the liver and colon of WT recipients (FIGS. 16A-16B). However, anti-IL-2-JES6 treatment did not increase the percentage of Tr1 cells in PD-L1^{-/-} recipients (FIG. 16C). The IL-10⁺ Tr1 cells were also IFN-γ⁺ but GM-CSF⁺ (FIG. 17).

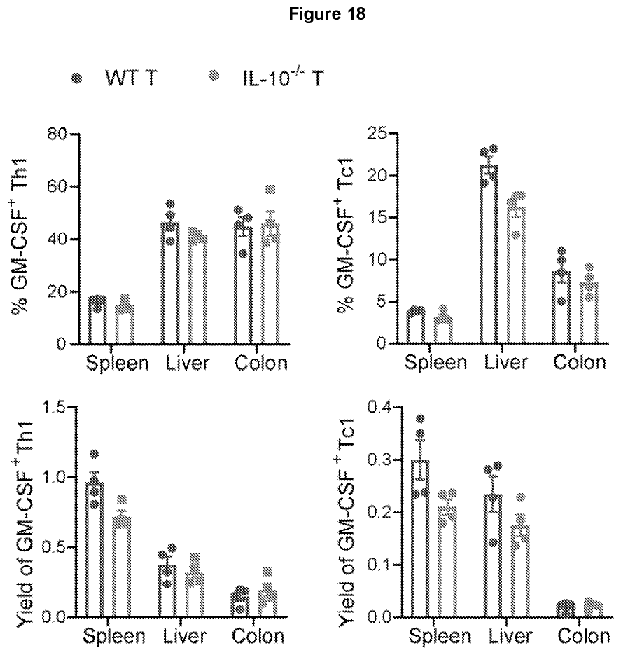

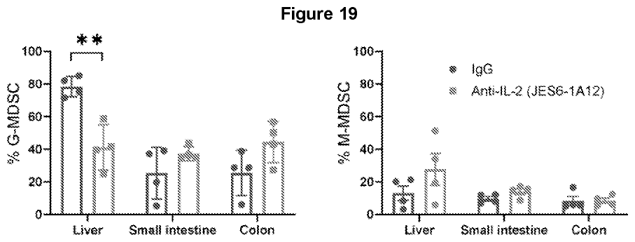

As compared to IgG treatment, anti-IL-2-JES6, but not anti-IL-2-S4B6, upregulated expression of Eomes and Blimp-1 by CD4⁺ T cells in the liver and colon of WT recipients (FIGS. 16D-16E). Anti-IL-2-JES6 did not upregulate expression of Eomes or Blimp-1 in PD-L1^{-/-} recipients (FIG. 16F). Finally, anti-IL-2-JES6 treatment did not prevent aGVHD induced by IL-10^{-/-} donor T cells (FIG. 16G); although IL-10 deficiency in donor T cells did not expand GM-CSF⁺ Th1 or Tc1 cells (FIG. 18). In addition, anti-IL-2-JES6 reduced the percentages of G-MDSC in the liver but not in the gut or on the percentages of M-MDSC in the gut or liver (FIG. 19). Therefore, anti-IL-2-JES6 treatment augments T cell expression of Eomes and Blimp-1 and T cell differentiation into IL-10-producing Tr1 cells in a tissue PD-L1-dependent manner; and that IL-10 from Tr1 cells also play an important role in anti-IL-2-JES6-mediated prevention of GVHD.

### Example 6. Prevention of GVHD by Tolerogenic Anti-IL-2-JES6 Requires Expression of PD-L1 by GVHD Target Tissues to Inhibit Activation of IL-2-Stat-5 and AKT-mTOR Pathways in Donor T Cells

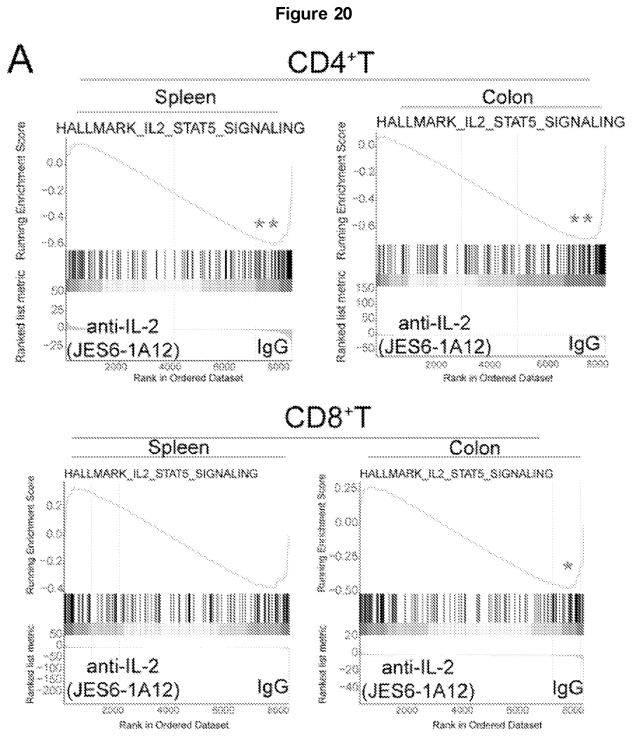

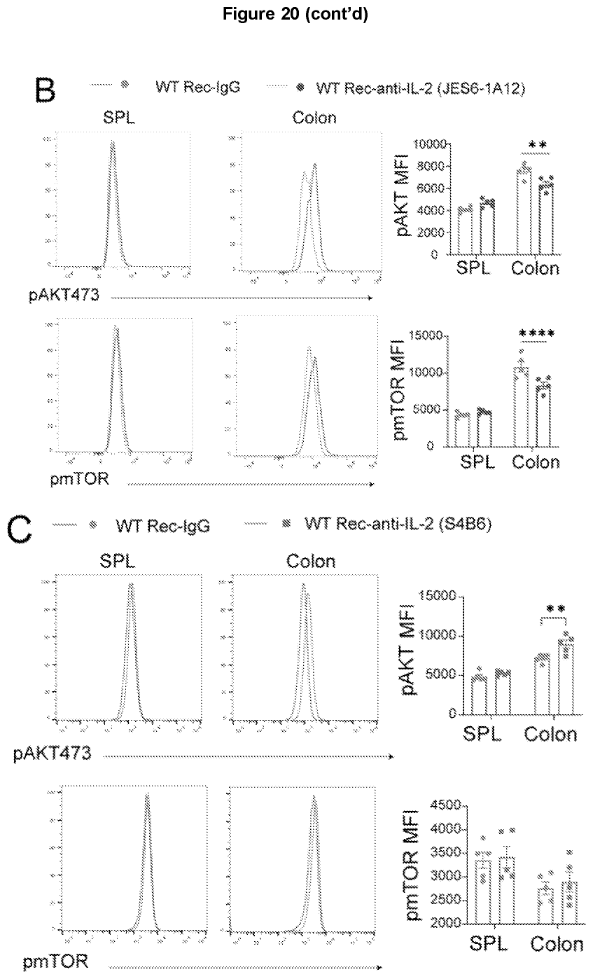

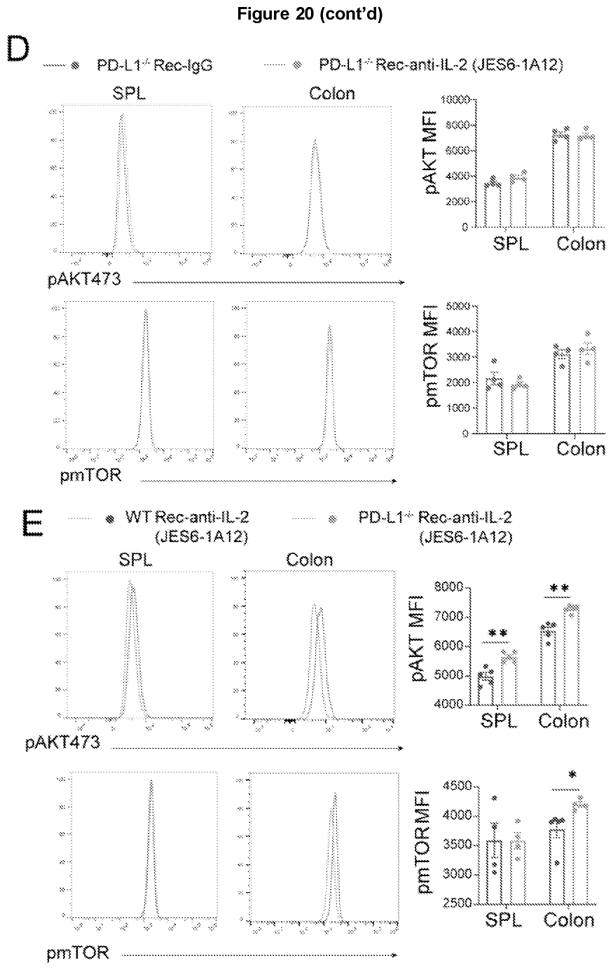

Gene Set Enrichment Analysis (GSEA) at day 6 showed inhibition of the IL-2-Stat5 pathway in the CD4⁺ T and CD8⁺ T cells from the spleen and colon of recipients treated with anti-IL-2-JES6 compared to control IgG (FIG. 20A). Anti-IL-2-JES6 but not anti-IL-2-S4B6 decreased the expression of pAKT and pmTOR in CD4⁺ T cells from the colon tissue but not the spleen of WT recipients (FIGS. 20B-20C). Anti-IL-2-JES6 treatment did not change pAKT or pmTOR expression in PD-L1^{-/-} recipients (FIG. 20D). Consistent with PD-1-mediated inhibition in WT recipients, AKT activation was higher in donor CD4⁺ T cells from the colon and spleen of anti-IL-2-JES6-treated PD-L1^{-/-} recipients than in anti-IL-2-JES6-treated WT recipients (FIG. 20E).

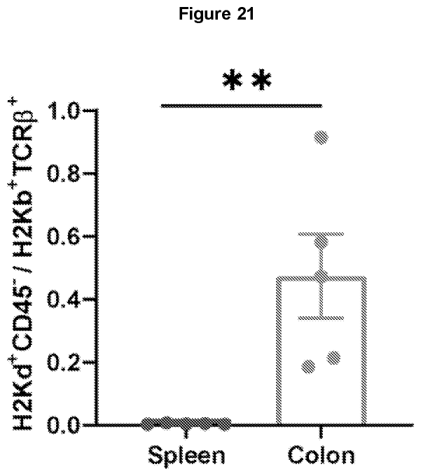

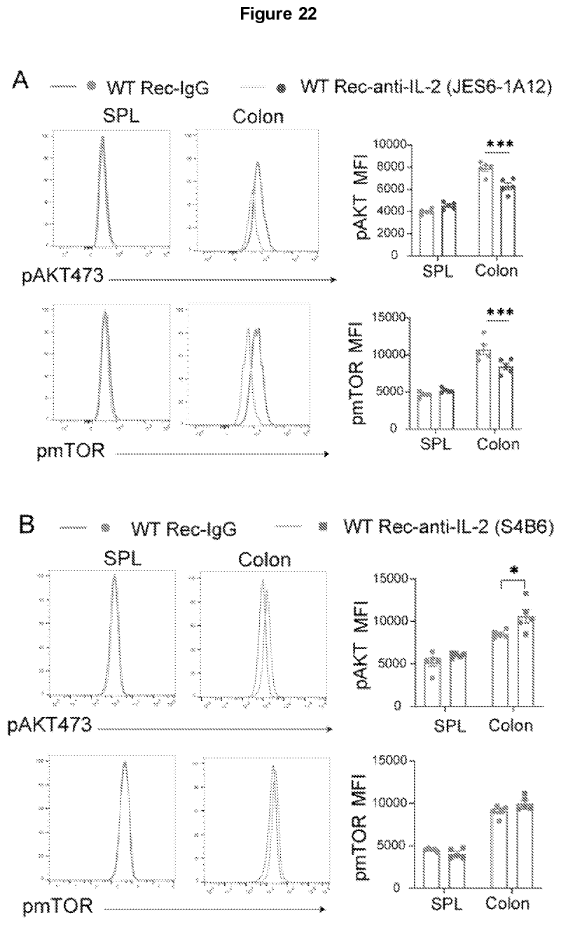

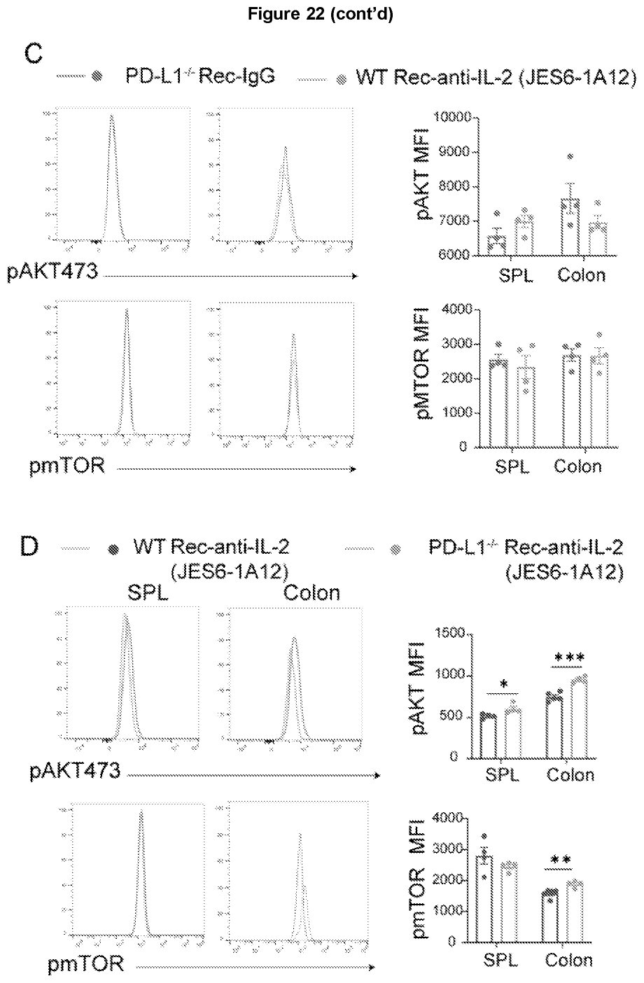

As shown in FIG. 20B, mTOR activation was not inhibited in WT CD4⁺ cells from the spleen of anti-IL-2-JES6-treated recipients, most likely reflecting the lack of PD-1 interaction with PD-L1 expressed by host parenchymal tissues, because the ratio of CD45⁻ parenchymal cells to donor T cells was more than 50-fold lower in the spleen than in the colon (FIG. 21). Similar results were observed with the CD8⁺ T cells (FIG. 22). Taken together, anti-IL-2-JES6 treatment inhibits IL-2-Stat5 signaling in donor T cells from both spleen and GVHD target tissues and inhibits AKT-mTOR signaling in donor T cells only in GVHD target tissues via PD-1 interaction with the tissue PD-L1.

### Example 7. Anti-IL-2-JES6 and Tacrolimus Induce Distinct T Cell Subpopulations and T Cell Transcriptional Signatures

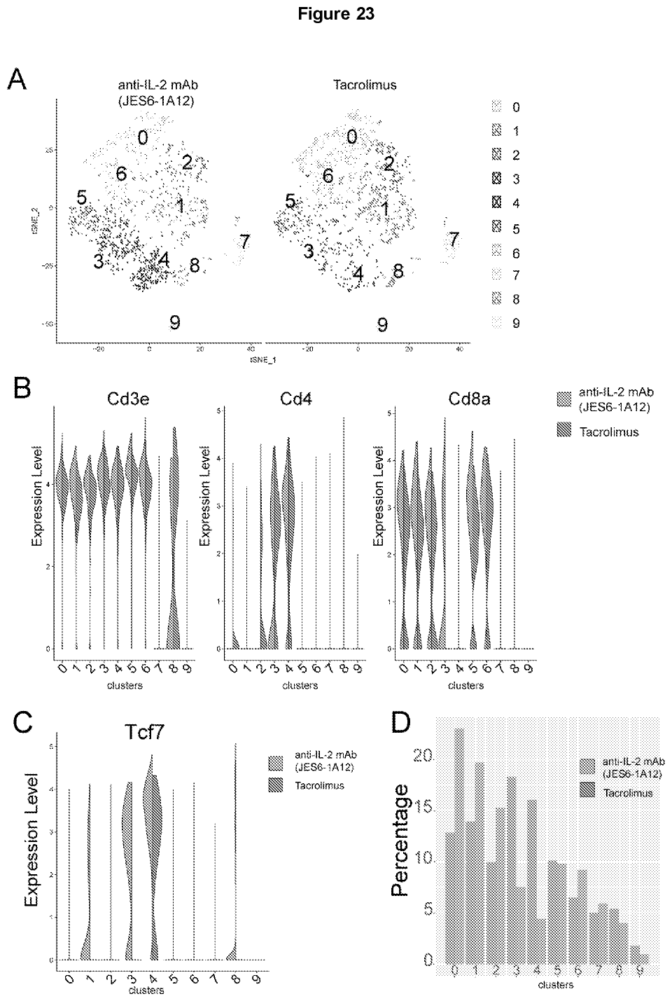

Anti-IL-2-JES6 treatment prevented aGVHD while preserving strong GVL effect that was markedly better than observed with TAC treatment (FIG. 2). Single cell RNA-seq was used to characterize the donor T cell subsets from the spleen of BCL1/luc tumor-bearing recipients treated with anti-IL-2-JES6 or TAC at day 7 after HCT. As visualized by t-distributed stochastic neighbor-embedding analysis (t-SNE), donor CD4⁺ and CD8⁺ T cells were grouped in 10 distinct clusters (FIG. 23A). Clusters 0, 1, 2, 5, 6 contain only CD8⁺ T cells, while Cluster 4 contains only CD4⁺ T cells. Cluster 3 contains both CD4⁺ and CD8⁺ T cells in anti-IL-2-JES6-treated recipient but only CD4⁺ T cells in TAC-treated recipients (FIG. 23B). The general cluster distribution of splenic T cells was similar in recipients treated with anti-IL-2-JES6 or TAC, and CD8⁺ T cells were more heterogeneous than CD4⁺ T cells.

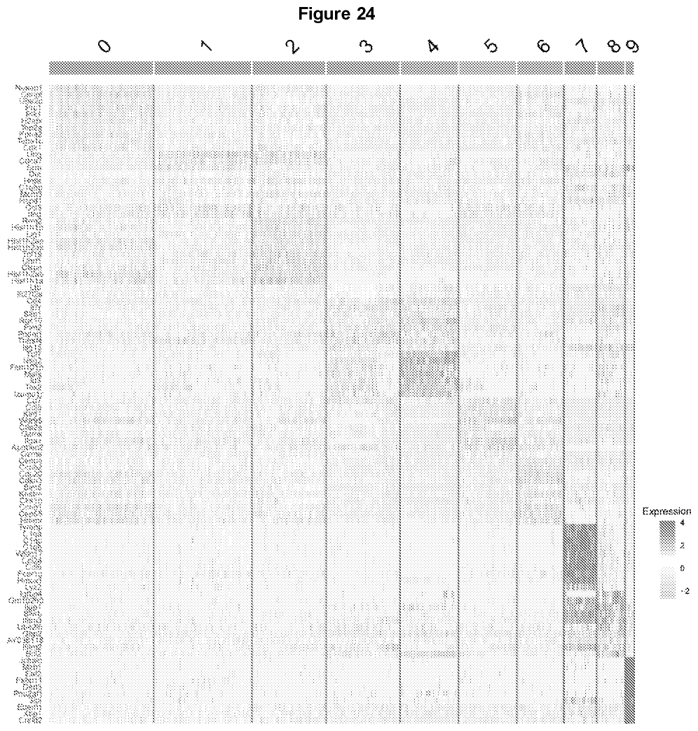

Gene expression profiles differed between the CD8⁺ T cell-enriched clusters. Clusters 0 and 6 had high expression of G2/M phase markers, while clusters 1 and 2 had high expression of S phase markers (FIG. 24). Cluster 3 had high expression of II7r, Tcf7 (encoding TCF-1) (FIG. 23C) and Bcl-2, with low expression of Gzma, Gzmk and markers of the cell cycle (FIG. 24), suggesting that this cluster is enriched for newly described TCF-1⁺ memory progenitor (Tmp) cells with unequivocal self-renewal potential [32]. Cluster 5 had high expression of Klrd1, Gzma, Gzmk, Id2, and Itgax, suggesting that Teff cells are enriched in this cluster (FIG. 24). Although the gene expression profiles of different clusters in anti-IL-2-JES6-treated and TAC-treated groups were similar, relative cluster sizes differed in the two groups. Clusters 0, 1 and 2 containing CD8⁺ T cells in S phase were larger in the TAC-treated group than in the anti-IL-2-treated group, while Clusters 3 and 4 containing TCF-1+CD4⁺ and CD8⁺ self-renewing memory progenitors were larger in the anti-IL-2-JES6-treated group than in the TAC group (FIGS. 23C-23D). TCF-1⁺ CD8⁺ T progenitors can further differentiate into cytolytic CD8⁺ T cells under CD4⁺ T cell help [32]. These results suggest that self-renewing CD8⁺ Tmp cells in lymphoid tissues are better preserved during anti-IL-2-JES6 treatment compared to TAC-treatment.

### Example 8. Anti-IL-2-JES6 Preserves CD8⁺ T Memory Progenitors and Functional Effectors that Mediate GVL Activity in Lymphoid Tissues More Effectively than Tacrolimus

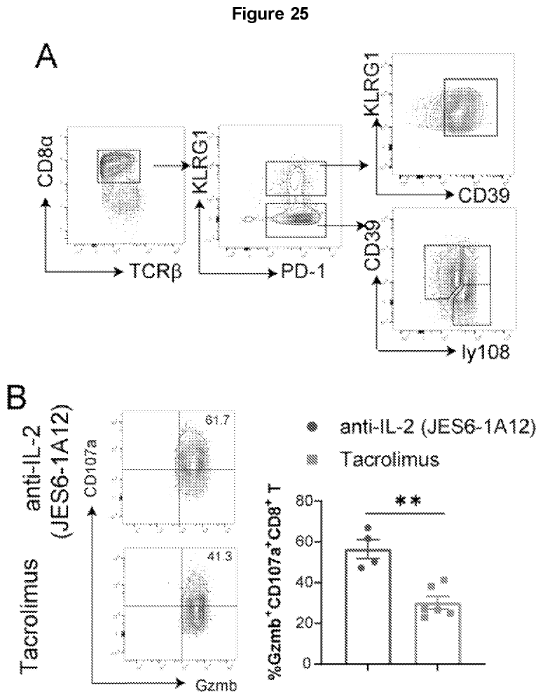

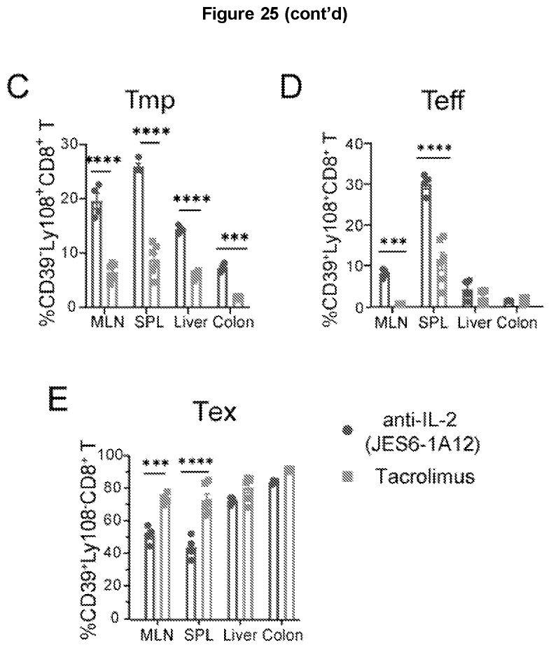

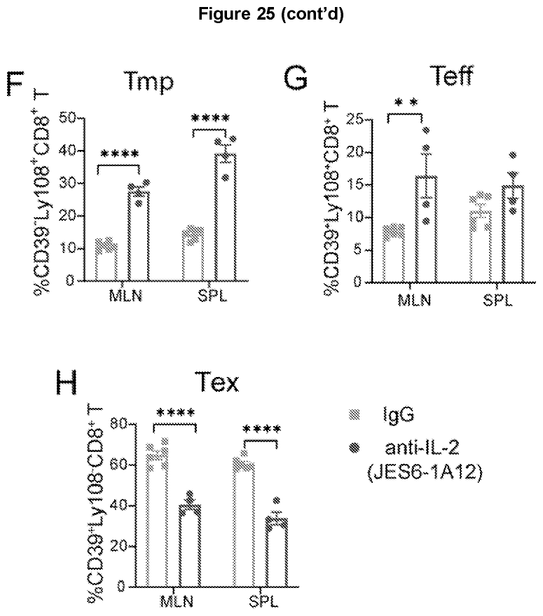

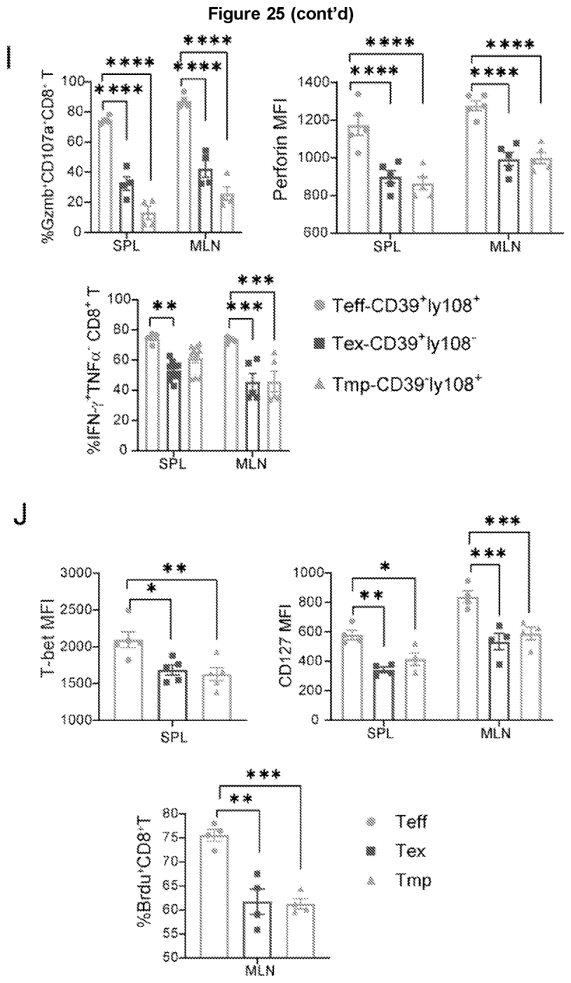

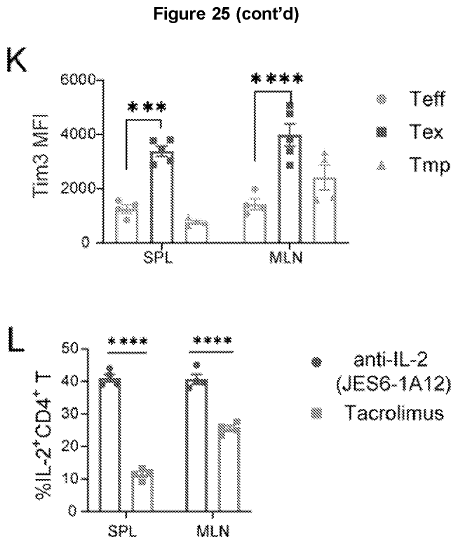

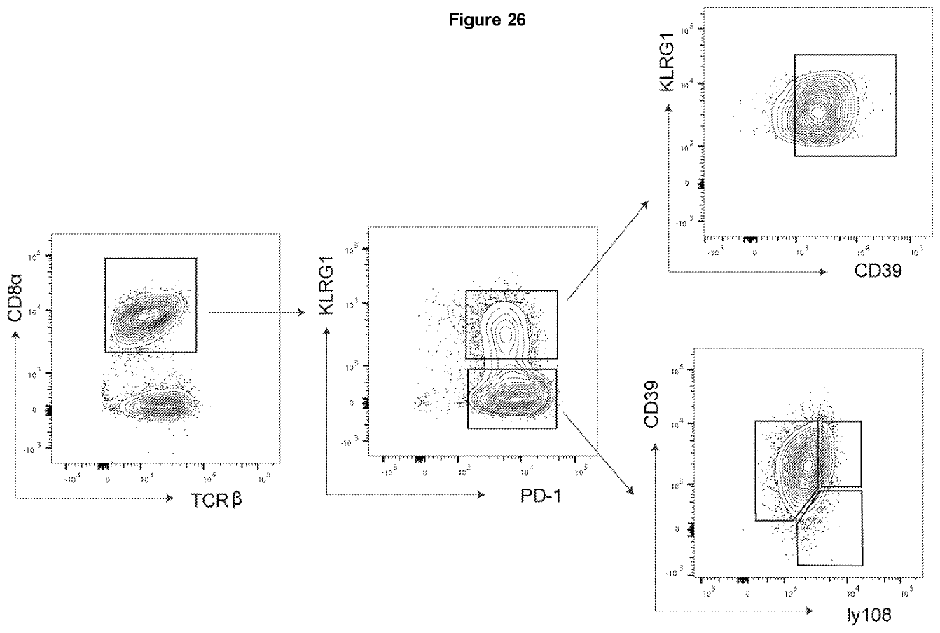

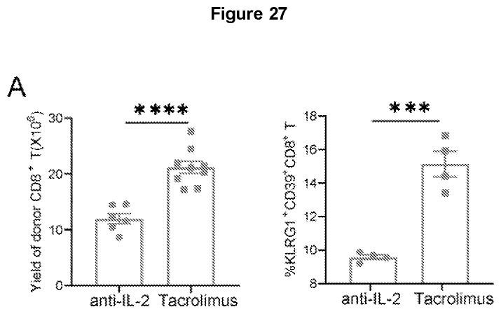

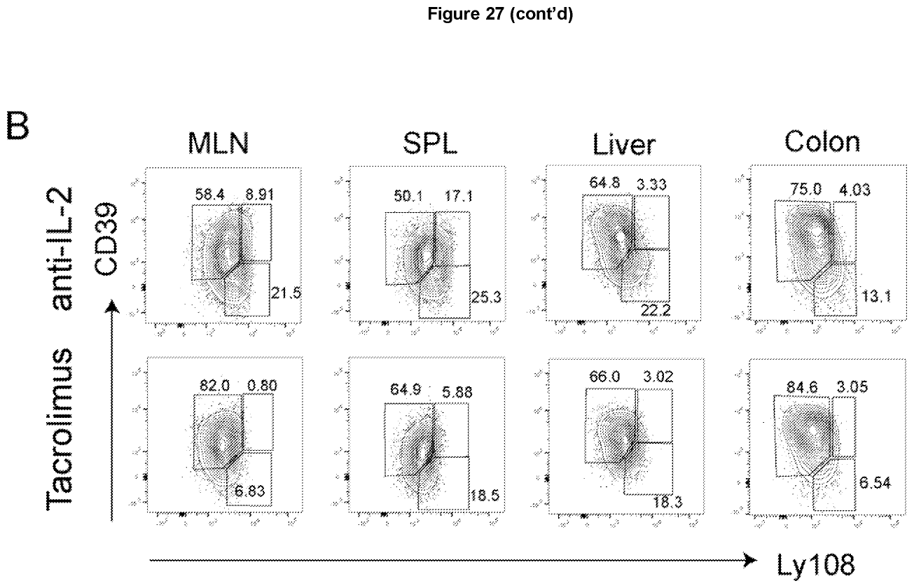

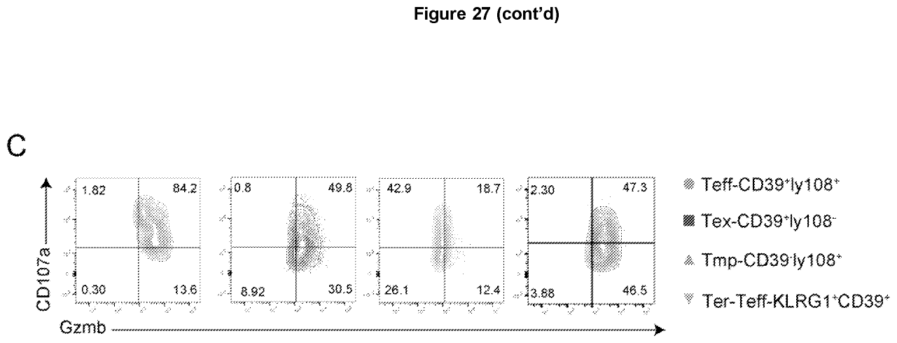

Ly108 can be used as surrogate to identify TCF-1⁺ CD8⁺ T progenitors [33] and CD8⁺ T cells can be divided into KLRG1⁺PD1⁺ and KLRG1⁻PD1⁺ subsets [33]. The KLRG1⁺PD-1⁺ subset are mostly KLRG1⁺ CD39⁺ terminally differentiated CD8⁺ T effectors (Ter-Teff) [33]. The KLRG1⁻PD-1⁺ subset can be further divided into CD39⁻ Ly108⁺ CD8⁺ Tmp, CD39⁺Ly108⁺ CD8⁺ T effector cells (Teff), and CD39⁺Ly108⁻ CD8⁺ exhausted T effector cells (Tex) [33]. All subsets were observed in the spleen of recipients treated with anti-IL-2 (FIG. 25A) or TAC (FIG. 26) at day 7 after HCT. As compared to TAC treatment, anti-IL-2-JES6-treatment reduced numbers of total donor CD8⁺ T cells and Ter-Teff cells in the spleen (FIG. 27A), but anti-IL-2-treatment markedly increased the percentage of CD107a⁺Granzyme B⁺ subset that have stronger cytolytic function among Ter-Teff cells (FIG. 25B).

Consistent with single cell RNA-seq analysis, as compared to TAC-treated recipients, the percentage of Tmp cells within the CD8⁺ T cell population was higher in the LN, SPL, liver and colon of anti-IL-2-JES6 treated recipients at day 7 after HCT than in TAC-treated recipients (FIGS. 25C and 27B). The percentage of Teff cells within the CD8⁺ population was higher in the LN and SPL of anti-IL-2-JES6-treated recipients than in TAC-treated recipients, with no differences in the liver or colon between the 2 groups (FIGS. 25D and 27B). The percentage of Tex cells within the CD8⁺ population in the LN and SPL of anti-IL-2-JES6-treated recipients was significantly lower than in TAC-treated recipients, with no difference in the liver or colon between the 2 groups (FIGS. 25E and 27B).

As compared to anti-IgG-treatment, anti-IL-2-JES6 treatment increased the percentages of Tmp and Teff cells within CD8⁺ cells in lymphoid tissues at day 7, while the percentage of Tex cells was lower in anti-IL-2-JES6-treated recipients than in IgG-treated controls (FIGS. 25F-25H). As compared with Teff cells, Tex cells and Tmp cells had lower expression of granzyme b, CD107a, perforin, IFN-γ, T-bet, CD127 and lower BrdU-labeling (FIGS. 25I-25J). Tim3 expression was higher in Tex than in Teff and Tmp (FIG. 25K). In addition, the percentage of IL-2-producing CD4⁺ T cells in SPL and LN was higher at day 7 after HCT in recipients treated with anti-IL-2-JES6 than in those treated with TAC (FIG. 25L). Taken together, these results indicate that anti-IL-2-JES6 treatment augments donor CD8⁺ T differentiation into Tmp cells and their Teff progeny; and that anti-IL-2-JES6 treatment is more effective than TAC-treatment for preserving the function of donor CD8⁺ T cells in the lymphoid tissues of recipients.

### REFERENCES

1. Li J M, Giver C R, Lu Y, Hossain M S, Akhtari M, Waller E K. Separating graft-versus-leukemia from graft-versus-host disease in allogeneic hematopoietic stem cell transplantation. Immunotherapy. 2009; 1(4):599-621.
2. Sung A D, Chao N J. Acute graft-versus-host disease: are we close to bringing the bench to the bedside? Best Pract Res Clin Haematol. 2013; 26(3):285-292.
3. Ebens C L, Maillard I. Notch signaling in hematopoietic cell transplantation and T cell alloimmunity. Blood Rev. 2013; 27(6):269-277.
4. Kanakry C G, Fuchs E J, Luznik L. Modern approaches to HLA-haploidentical blood or marrow transplantation. Nat Rev Clin Oncol. 2016; 13(1):10-24.
5. Negrin R S. Graft-versus-host disease versus graft-versus-leukemia. Hematology Am Soc Hematol Educ Program. 2015; 2015:225-230.
6. Chang Y J, Zhao X Y, Huang X J. Strategies for Enhancing and Preserving Anti-leukemia Effects Without Aggravating Graft-Versus-Host Disease. Front Immunol. 2018; 9:3041.
7. Sofi M H, Wu Y, Schutt S D, et al. Thioredoxin-1 confines T cell alloresponse and pathogenicity in graft-versus-host disease. J Clin Invest. 2019; 129(7):2760-2774.
8. Choi J, Cooper M L, Staser K, et al. Baricitinib-induced blockade of interferon gamma receptor and interleukin-6 receptor for the prevention and treatment of graft-versus-host disease. Leukemia. 2018; 32(11):2483-2494.
9. Butte M J, Keir M E, Phamduy T B, Sharpe A H, Freeman G J. Programmed death-1 ligand 1 interacts specifically with the B7-1 costimulatory molecule to inhibit T cell responses. Immunity. 2007; 27(1):111-122.
10. Cassady K, Martin P J, Zeng D. Regulation of GVHD and GVL Activity via PD-L1 Interaction With PD-1 and CD80. Front Immunol. 2018; 9:3061.
11. Freeman G J, Long A J, Iwai Y, et al. Engagement of the PD-1 immunoinhibitory receptor by a novel B7 family member leads to negative regulation of lymphocyte activation. J Exp Med. 2000; 192(7):1027-1034.
12. Sharpe A H, Pauken K E. The diverse functions of the PD1 inhibitory pathway. Nat Rev Immunol. 2018; 18(3):153-167.
13. Ni X, Song Q, Cassady K, et al. PD-L1 interacts with CD80 to regulate graft-versus-leukemia activity of donor CD8+ T cells. J Clin Invest. 2017; 127(5):1960-1977.
14. Carter L, Fouser L A, Jussif J, et al. PD-1:PD-L inhibitory pathway affects both CD4(+) and CD8(+) T cells and is overcome by IL-2. Eur J Immunol. 2002; 32(3):634-643.
15. Sykes M, Romick M L, Sachs D H. Interleukin 2 prevents graft-versus-host disease while preserving the graft-versus-leukemia effect of allogeneic T cells. Proc Nat/Acad Sci USA. 1990; 87(15):5633-5637.
16. Andolfi G, Fousteri G, Rossetti M, et al. Enforced IL-10 expression confers type 1 regulatory T cell (Tr1) phenotype and function to human CD4(+) T cells. Mol Ther. 2012; 20(9):1778-1790.
17. Zhang P, Lee J S, Gartlan K H, et al. Eomesodermin promotes the development of type 1 regulatory T (TR1) cells. Sci Immunol. 2017; 2(10).
18. Blazar B R, MacDonald KPA, Hill G R. Immune regulatory cell infusion for graft-versus-host disease prevention and therapy. Blood. 2018; 131(24):2651-2660.
19. Schneidawind D, Pierini A, Negrin R S. Regulatory T cells and natural killer T cells for modulation of GVHD following allogeneic hematopoietic cell transplantation. Blood. 2013; 122(18):3116-3121.
20. Koreth J, Matsuoka K, Kim H T, et al. Interleukin-2 and regulatory T cells in graft-versus-host disease. N Engl J Med. 2011; 365(22):2055-2066.
21. Hongo D, Tang X, Dutt S, Nador R G, Strober S. Interactions between NKT cells and Tregs are required for tolerance to combined bone marrow and organ transplants. Blood. 2012; 119(6):1581-1589.
22. Xhaard A, Moins-Teisserenc H, Busson M, et al. Reconstitution of regulatory T-cell subsets after allogeneic hematopoietic SCT. Bone Marrow Transplant. 2014; 49(8):1089-1092.
23. Amarnath S, Mangus C W, Wang J C, et al. The PDL1-PD1 axis converts human TH1 cells into regulatory T cells. Sci Transl Med. 2011; 3(111):111ra120.
24. Zhang Y, Joe G, Hexner E, Zhu J, Emerson S G. Host-reactive CD8⁺ memory stem cells in graft-versus-host disease. Nat Med. 2005; 11(12):1299-1305.
25. He S, Liu Y, Meng L, et al. Ezh2 phosphorylation state determines its capacity to maintain CD8(+) T memory precursors for antitumor immunity. Nat Commun. 2017; 8(1):2125.
26. Spangler J B, Tomala J, Luca V C, et al. Antibodies to Interleukin-2 Elicit Selective T Cell Subset Potentiation through Distinct Conformational Mechanisms. Immunity. 2015; 42(5):815-825.
27. Boyman O, Kovar M, Rubinstein M P, Surh C D, Sprent J. Selective stimulation of T cell subsets with antibody-cytokine immune complexes. Science. 2006; 311(5769):1924-1927.
28. Matte-Martone C, Venkatesan S, Tan H S, et al. Graft-versus-leukemia (GVL) against mouse blast-crisis chronic myelogenous leukemia (BC-CML) and chronic-phase chronic myelogenous leukemia (CP-CML): shared mechanisms of T cell killing, but programmed death ligands render CP-CML and not BC-CML GVL resistant. J Immunol. 2011; 187(4):1653-1663.
29. Wherry E J, Kurachi M. Molecular and cellular insights into T cell exhaustion. Nat Rev Immunol. 2015; 15(8):486-499.
30. Tugues S, Amorim A, Spath S, et al. Graft-versus-host disease, but not graft-versus-leukemia immunity, is mediated by G M-CSF-licensed myeloid cells. Sci Transl Med. 2018; 10(469).
31. Gartlan K H, Koyama M, Lineburg K E, et al. Donor T-cell-derived G M-CSF drives alloantigen presentation by dendritic cells in the gastrointestinal tract. Blood Adv. 2019; 3(19):2859-2865.
32. Zander R, Schauder D, Xin G, et al. CD4(+) T Cell Help Is Required for the Formation of a Cytolytic CD8(+) T Cell Subset that Protects against Chronic Infection and Cancer. Immunity. 2019; 51(6):1028-1042 e1024.
33. Chen Z, Ji Z, Ngiow S F, et al. TCF-1-Centered Transcriptional Network Drives an Effector versus Exhausted CD8 T Cell-Fate Decision. Immunity. 2019; 51(5):840-855 e845.
34. Gallon L, Traitanon O, Yu Y, et al. Differential Effects of Calcineurin and Mammalian Target of Rapamycin Inhibitors on Alloreactive Th1, Th17, and Regulatory T Cells. Transplantation. 2015; 99(9):1774-1784.
35. Rosenbeck L L, Kiel P J, Kalsekar I, et al. Prophylaxis with sirolimus and tacrolimus+/−antithymocyte globulin reduces the risk of acute graft-versus-host disease without an overall survival benefit following allogeneic stem cell transplantation. Biol Blood Marrow Transplant. 2011; 17(6):916-922.
36. Malek T R, Castro I. Interleukin-2 receptor signaling: at the interface between tolerance and immunity. Immunity. 2010; 33(2):153-165.
37. Zheng H, Matte-Martone C, Li H, et al. Effector memory CD4+ T cells mediate graft-versus-leukemia without inducing graft-versus-host disease. Blood. 2008; 111(4):2476-2484.
38. Zheng H, Matte-Martone C, Jain D, McNiff J, Shlomchik W D. Central memory CD8+ T cells induce graft-versus-host disease and mediate graft-versus-leukemia. J Immunol. 2009; 182(10):5938-5948.
39. Zhang P, Wu J, Deoliveira D, Chao N J, Chen B J. Allospecific CD4(+) effector memory T cells do not induce graft-versus-host disease in mice. Biol Blood Marrow Transplant. 2012; 18(10):1488-1499.
40. Bleakley M, Heimfeld S, Loeb K R, et al. Outcomes of acute leukemia patients transplanted with naive T cell-depleted stem cell grafts. J Clin Invest. 2015; 125(7):2677-2689.
41. Trotta E, Bessette P H, Silveria S L, et al. A human anti-IL-2 antibody that potentiates regulatory T cells by a structure-based mechanism. Nat Med. 2018; 24(7):1005-1014.
42. Chakraverty R, Cote D, Buchli J, et al. An inflammatory checkpoint regulates recruitment of graft-versus-host reactive T cells to peripheral tissues. J Exp Med. 2006; 203(8):2021-2031.
43. Yu G, Wang L G, Han Y, He Q Y. clusterProfiler: an R package for comparing biological themes among gene clusters. OMICS. 2012; 16(5):284-287.

1. A method of preventing or treating GVHD while preserving GVL activity in a subject receiving a hematopoietic cell transplantation (HCT) comprising administering to the subject an effective amount of an anti-IL-2 antibody.

2. The method of claim 1, wherein the subject receives an allogeneic HCT.

3. The method of claim 1 or claim 2, wherein the GVHD is acute GVHD.

4. The method of any one of claims 1-3, wherein the anti-IL-2 antibody is a monoclonal antibody.

5. The method of any one of claims 1-4, wherein the anti-IL-2 antibody is a recombinant antibody.

6. The method of any one of claims 1-5, wherein the anti-IL-2 antibody is a human antibody.

7. The method of any one of claims 1-5, wherein the anti-IL-2 antibody is a humanized antibody.

8. The method of any one of claims 1-7, wherein the anti-IL-2 antibody is anti-IL-2-JES6 antibody.

9. The method of any one of claims 1-8, wherein the anti-IL-2 antibody is administered to the subject on the same day of receiving HCT.

10. The method of any one of claims 1-8, the anti-IL-2 antibody is administered to the subject after receiving HCT.

11. The method of any one of claims 1-9, wherein multiple doses of the anti-IL-2 antibody are administered immediately after HCT.

12. The method of any one of claims 1-11, a single dose of the anti-IL-2 antibody is administered each day.

13. The method of any one of claims 1-12, wherein the anti-IL-2 antibody is administered every other day fora week, for two weeks, for three weeks, or for a month after HCT.

14. The method of any one of claims 1-13, wherein the subject is human.

15. A composition comprising an effective amount of an anti-IL-2 antibody for preventing or treating GVHD while preserving GVL activity in a subject receiving a hematopoietic cell transplantation (HCT).

16. The composition of claim 15, wherein the anti-IL-2 antibody is a monoclonal antibody, a recombinant antibody, a human antibody, or a humanized antibody.

17. The composition of claim 15 or claim 16, wherein the anti-IL-2 antibody is anti-IL-2-JES6 antibody.

18. Use of an anti-IL-2 antibody for the manufacture of a medicament for preventing or treating GVHD while preserving GVL activity in a subject receiving a hematopoietic cell transplantation (HCT).

19. The use of claim 18, wherein the anti-IL-2 antibody is a monoclonal antibody, a recombinant antibody, a human antibody, or a humanized antibody.

20. The use of claim 18 or claim 19, wherein the anti-IL-2 antibody is anti-IL-2-JES6 antibody.
